(19) United States
(12) Patent Application Publication
(10) Pub. No.: US 2023/0357387 A1
(43) Pub. Date: Nov. 9, 2023

(54) ZIP12 ANTIBODY

(71) Applicant: IP2IPO INNOVATIONS LIMITED, London (GB)

(72) Inventors: Stephanie HOPLEY, Cambridge (GB); Paul HAMBLIN, Cambridge (GB); Martin WILKINS, London (GB); Lan ZHAO, London (GB)

(21) Appl. No.: 18/028,498

(22) PCT Filed: Sep. 24, 2021
(86) PCT No.: PCT/GB2021/052495

§ 371 Date: Mar. 24, 2023

(30) Foreign Application Priority Data
Sep. 24, 2020 (GB) 2015115.5

(51) Int. Cl. C07K 16/28

(52) U.S. Cl. CPC C07K 16/28; C07K 2317/33; C07K 2317/34; C07K 2317/76; C07K 2317/24

(57) ABSTRACT

The invention relates to ZIP12 antibodies. The invention extends to compositions comprising the antibodies, including pharmaceutical compositions and kits. The invention also extends to methods of making and using the antibodies, for example in therapy and diagnosis of hypoxia-related diseases, such as pulmonary hypertension and cancer.

The invention relates to ZIP12 antibodies. The invention extends to compositions comprising the antibodies, including pharmaceutical compositions and kits. The invention also extends to methods of making and using the antibodies, for example in therapy and diagnosis of hypoxia-related diseases, such as pulmonary hypertension and cancer.

Pulmonary hypertension (PH) is a pathological condition characterised by elevated pulmonary artery pressure and structurally remodelled pulmonary vessels. The current treatments for pulmonary hypertension centre on the pharmacological manipulation of signalling mechanisms used by vasoactive factors and have limited therapeutic benefit. There exists a significant unmet medical need for new therapeutics and diagnostics for this potentially lethal condition.

The typical response of the adult mammalian pulmonary circulation to a low oxygen environment is vasoconstriction and structural remodelling of pulmonary arterioles, leading to chronic elevation of pulmonary artery pressure (pulmonary hypertension) and right ventricular hypertrophy. Some mammals, however, exhibit genetic resistance to hypoxia-induced pulmonary hypertension (WILKINS, M. R. et al., Pathophysiology and treatment of high-altitude pulmonary vascular disease. Circulation. 2015, 131, 582-590; ZHAO, L. et al., Right ventricular hypertrophy secondary to pulmonary hypertension is linked to rat chromosome 17: evaluation of cardiac ryanodine Ryr2 receptor as a candidate. Circulation. 2001, 103, 442-447; RHODES, J., Comparative physiology of hypoxic pulmonary hypertension: historical clues from brisket disease. Journal of applied physiology. 2005, 98, 1092-1100). The inventors have previously reported that the Fisher 344 (F344) rat strain is resistant to hypoxia-induced pulmonary hypertension compared to the Wistar Kyoto (WKY) strain (ZHAO, L. et al., Right ventricular hypertrophy secondary to pulmonary hypertension is linked to rat chromosome 17: evaluation of cardiac ryanodine Ryr2 receptor as a candidate. Circulation. 2001, 103, 442-447). However, the cause of this resistance has never been identified.

The inventors have also previously utilised a congenic breeding program and comparative genomics to exploit this variation in the rat and have identified the gene, Slc39a12, as a major regulator of hypoxia-induced pulmonary vascular remodelling. Slc39a12 encodes the zinc transporter, ZIP12. They found that ZIP12 expression is increased in many cell types, including endothelial, smooth muscle and interstitial cells, in the remodelled pulmonary arterioles of rats, cows and humans susceptible to hypoxia-induced pulmonary hypertension. The inventors have shown that ZIP12 expression in pulmonary vascular smooth muscle cells is hypoxia-dependent and that targeted inhibition of ZIP12 inhibits the rise in intracellular labile zinc in hypoxia-exposed pulmonary vascular smooth muscle cells and their proliferation in culture. The inventors have also previously demonstrated that genetic disruption of ZIP12 expression attenuates the development of pulmonary hypertension in rats housed in a hypoxic atmosphere.

However, to date no specific treatment is available for targeting ZIP12 protein, which the inventors believe may provide for an improved means of treating pulmonary hypertension. Furthermore, diagnosis of pulmonary hypertension often requires invasive procedures, such as right heart catheterisation and echocardiograms. Thus, identification of new markers of pulmonary hypertension that could be measured through a simple and less invasive procedure is also desirable.

The inventors hypothesized that ZIP12 inhibition could prevent hypoxia-induced Pulmonary Vascular Smooth Muscle Cells (PVSMC) proliferation in vitro and pulmonary angiogenesis ex vivo. This identifies ZIP12 as a potential new therapeutic target for the treatment of the underlying disease mechanisms of pulmonary hypertension, which has led to the inventor's further work in developing antibodies that are capable of targeting the extracellular domain of ZIP12 to inhibit its function. The anti-ZIP12 activity of these antibodies means that they are useful as therapeutic agents in their own right, and may be used in the treatment, amelioration or prevention of any hypoxia-induced or hypoxia-associated condition, and in particular, though not exclusively, pulmonary hypertension.

Accordingly, in a first aspect of the invention, there is provided an antibody or antigen-binding fragment thereof that specifically binds to an extracellular region of ZIP12.

As shown in the examples, the inventors have identified the extracellular region or domain of the ZIP12 protein as being key to its function and have therefore developed antibodies that are capable of binding to, and inhibiting, ZIP12 function. For example, as shown in FIGS. 2 and 3, the inventors have developed a large number of antibodies that they have demonstrated specially target the extracellular domain and inhibit ZIP12 function. Furthermore, as shown in FIGS. 6, 7 and 9 the inventors have demonstrated that not only do the antibodies inhibit ZIP12 function, they do not target the ZIP4 protein.

Preferably, the antibody or antigen binding fragment thereof of the invention is capable of inhibiting ZIP12 function. Preferably, the antibody or antigen binding fragment thereof of the invention is capable of inhibiting ZIP12 function such that the rise in intracellular labile zinc in hypoxia-exposed pulmonary vascular smooth muscle cells and their proliferation in culture is inhibited.

ZIP12, ZIP13 and ZIP4 are all zinc transporters. However, ZIP4 is not involved in the pathogenesis of pulmonary hypertension but does have a significant role in tissue homeostasis, metabolism, development and immunity. Furthermore, ZIP13 has been shown to regulate intracellular zinc and smad signalling, which is important for BMP/TGF-beta signalling (see Fakuda et al, J Biol Inorg Chem, 2011). Impaired BMP signalling is known to be linked to pulmonary hypertension, as shown in Morrell et al Nature Reviews Cardiology, 2015. Therefore, the inhibition of ZIP13, leading to lower zinc concentrations, may impair BMP signalling and cause pulmonary hypertension, not cure it.

Therefore, it is important that the antibodies of the invention which target ZIP12 do so specifically, and have no or little cross-reactivity with ZIP13 and/or ZIP4, because this could result in significant unwanted off-target effects.

Accordingly, preferably the antibody or antigen binding fragment thereof of the invention does not substantially bind to human ZIP13. Preferably, the antibody or antigen binding fragment thereof of the invention has no cross-reactivity with human ZIP13.

In addition, preferably the antibody or antigen binding fragment thereof of the invention does not substantially bind to human ZIP4. Preferably, the antibody or antigen binding fragment thereof of the invention has no cross-reactivity with human ZIP4.

In one embodiment, ZIP12 may be represented by Genbank ID No: NP-001138667, which is provided herein as SEQ ID No: 1, as follows:

[SEQ ID No: 1]
MCFRTKLSVSWVPLFLLLSRVFSTETDKPSAQDSRSRGSSGQPADLLQVL
SAGDHPPHNHSRSLIKTLLEKTGCPRRRNGMQGDCNLCFEPDALLLIAGG
NFEDQLREEVVQRVSLLLLYYIIHQEEICSSKLNMSNKEYKFYLHSLLSL
RQDEDSSFLSQNETEDILAFTRQYFDTSQSQCMETKTLQKKSGIVSSEGA
NESTLPQLAAMIITLSLQGVCLGQGNLPSPDYFTEYIFSSLNRTNTLRLS
ELDQLLNTLWTRSTCIKNEKIHQFQRKQNNIITHDQDYSNFSSSMEKESE
DGPVSWDQTCFSARQLVEIFLQKGLSLISKEDFKQMSPGIIQQLLSCSCH
LPKDQQAKLPPTTLEKYGYSTVAVTLLTLGSMLGTALVLFHSCEENYRLI
LQLFVGLAVGTLSGDALLHLIPQVLGLHKQEAPEFGHFHESKGHIWKLMG
LIGGIHGFFLIEKCFILLVSPNDKQGLSLVNGHVGHSHHLALNSELSDQA
GRGKSASTIQLKSPEDSQAAEMPIGSMTASNRKCKAISLLAIMILVGDSL
HNFADGLAIGAAFSSSSESGVTTTIAILCHEIPHEMGDFAVLLSSGLSMK
TAILMNFISSLTAFMGLYIGLSVSADPCVQDWIFTVTAGMFLYLSLVEML
PEMTHVQTQRPWMMFLLQNFGLILGWLSLLLLAIYEQNIKI

The antibody or antigen-binding fragment thereof may bind to a region between amino acid positions 1 and 202 of SEQ ID No: 1, which corresponds to the extracellular domain of ZIP12.

Thus, preferably the antibody or antigen-binding fragment thereof may bind to one or more amino acids between amino acid positions 1 and 202 of ZIP12, which is provided herein as SEQ ID No: 2, as follows:

[SEQ ID No: 2]
MCFRTKLSVSWVPLFLLLSRVFSTETDKPSAQDSRSRGSSGQPADLLQVL
SAGDHPPHNHSRSLIKTLLEKTGCPRRRNGMQGDCNLCFEPDALLLIAGG
NFEDQLREEVVQRVSLLLLYYIIHQEEICSSKLNMSNKEYKFYLHSLLSL
RQDEDSSFLSQNETEDILAFTRQYFDTSQSQCMETKTLQKKSGIVSSEGA
NE

Thus, preferably the antibody or antigen-binding fragment thereof binds to an epitope within a sequence comprising or consisting of a sequence as substantially set out in SEQ ID No: 2, or a variant or fragment thereof.

Preferably, the antibody or antigen-binding fragment thereof binds to one or more amino acids in SEQ ID No: 2, or a fragment or variant thereof. Preferably, the antibody or antigen-binding fragment thereof binds to any 5, 10, 15, 20, 25, 30, 35, 40 or 45 amino acid sequence present in SEQ ID No: 2, or a variant or fragment thereof.

In one embodiment, the antibody or antigen-binding fragment thereof may bind to one or more amino acid between amino acid positions 20 and 202 of SEQ ID No: 2. The antibody or antigen-binding fragment thereof may bind to one or more amino acid between amino acid positions 20 and 180, or between amino acid positions 20 and 160, or between amino acid positions 20 and 140, or between amino acid positions 20 and 120, or between amino acid positions 20 and 110, or between amino acid positions 20 and 108 of SEQ ID No: 2. The antibody or antigen-binding fragment thereof may bind to one or more amino acid between amino acid positions 20 and 104 of SEQ ID No: 2.

The antibody or antigen-binding fragment thereof may bind to one or more amino acid between amino acid positions 40 and 202, or between amino acid positions 60 and 202, or between amino acid positions 80 and 202, or between amino acid positions 100 and 202, or between amino acid positions 120 and 202, or between amino acid positions 140 and 202, or between amino acid positions 150 and 202 of SEQ ID No: 2. The antibody or antigen-binding fragment thereof may bind to one or more amino acid between amino acid positions 156 and 202 of SEQ ID No: 2.

The epitope may be linear or conformational. The term “linear epitope” can mean an epitope consisting of amino acid residues that form a sequence together in the primary sequence of the protein antigen, i.e. sequential or continuous epitope. The term “conformational epitope” can mean an epitope consisting of amino acid residues, at least some of which are separated from others in the primary sequence of the protein antigen, but which together assemble in the 3D structure and are recognised by an antibody, i.e. discontinuous epitope.

In one embodiment, ZIP4 may be represented by Genbank ID No: NP-570901, which is provided herein as SEQ ID No: 19, as follows:

[SEQ ID No: 19]
MASLVSLELGLLLAVLVVTATASPPAGLLSLLTSGQGALDQEALGGLLN
TLADRVHCANGPCGKCLSVEDALGLGEPEGSGLPPGPVLEARYVARLSA
AAVLYLSNPEGTCEDARAGLWASHADHLLALLESPKALTPGLSWLLQRM
QARAAGQTPKTACVDIPQLLEEAVGAGAPGSAGGVLAALLDHVRSGSCF
HALPSPQYFVDFVFQQHSSEVPMTLAELSALMQRLGVGREAHSDHSHRH
RGASSRDPVPLISSSNSSSVWDTVCLSARDVMAAYGLSEQAGVTPEAWA
QLSPALLQQQLSGACTSQSRPPVQDQLSQSERYLYGSLATLLICLCAVF
GLLLLTCTGCRGVTHYILQTFLSLAVGALTGDAVLHLTPKVLGLHTHSE
EGLSPQPTWRLLAMLAGLYAFFLFENLFNLLLPRDPEDLEDGPCGHSSH
SHGGHSHGVSLQLAPSELRQPKPPHEGSRADLVAEESPELLNPEPRRLS
PELRLLPYMITLGDAVHNFADGLAVGAAFASSWKTGLATSLAVFCHELP
HELGDFAALLHAGLSVRQALLLNLASALTAFAGLYVALAVGVSEESEAW
ILAVATGLFLYVALCDMLPAMLKVRDPRPWLLFLLHNVGLLGGWTVLLL
LSLYEDDITF

Thus, preferably the antibody or antigen-binding fragment thereof does not bind to a sequence as substantially set out in SEQ ID No: 19, or a variant or fragment thereof.

In one embodiment, ZIP13 may be represented by Gene ID No: 91252, which is provided herein as SEQ ID No: 75, as follows:

[SEQ ID No: 75]
MPGCPCPGCGMAGPRLLFLTALALELLERAGGSQPALRSRGTATACRLD
NKESESWGALLSGERLDTWICSLLGSLMVGLSGVFPLLVIPLEMGTMLR
SEAGAWRLKQLLSFALGGLLGNVFLHLLPEAWAYTCSASPGGEGQSLQQ
QQQLGLWVIAGILTFLALEKMFLDSKEEGTSQAPNKDPTAAAAALNGGH
CLAQPAAEPGLGAVVRSIKVSGYLNLLANTIDNFTHGLAVAASFLVSKK
IGLLTTMAILLHEIPHEVGDFAILLRAGFDRWSAAKLQLSTALGGLLGA
GFAICTQSPKGVVGCSPAAEETAAWVLPFTSGGFLYIALVNVLPDLLEE
EDPWRSLQQLLLLCAGIVVMVLFSLFVD

Thus, preferably the antibody or antigen-binding fragment thereof does not bind to a sequence as substantially set out in SEQ ID No: 75, or a variant or fragment thereof.

The invention extends to both whole antibodies (i.e. immunoglobulins) with immunospecificity for the extracellular portion of ZIP12, as well as to antigen-binding fragments or regions of the corresponding full-length antibody.

The antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers. The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. the extracellular portion of ZIP12, or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The heavy chain constant region typically comprises three domains, C_{H1}, C_{H2}, and C_{H3}. Each light chain typically comprises a light chain variable region (VL) and a light chain constant region. The light chain constant region typically comprises one domain, abbreviated CL.

Each heavy chain and light chain generally comprise three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The light chain from any vertebrate species can be assigned to one of two types, called kappa and lambda, based on the sequence of the constant domain.

The constant region consists of one of five heavy chain sequences (μ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

In one preferred embodiment, the antibody or antigen-binding fragment thereof comprises a polyclonal antibody, or an antigen-binding fragment thereof. The antibody or antigen-binding fragment thereof may be generated in a rabbit, mouse or rat.

Preferably, the antibody or antigen-binding fragment thereof is obtained by immunising a host animal with the extracellular portion of ZIP12, or a variant or fragment thereof, and then collecting the antibody or antigen-binding fragment thereof. The host animal may be a rabbit.

In another preferred embodiment, the antibody or antigen-binding fragment thereof comprises a monoclonal antibody or an antigen-binding fragment thereof. Preferably, the antibody of the invention is a human antibody. As used herein, the term “human antibody” can mean an antibody, such as a monoclonal antibody, which comprises substantially the same heavy and light chain CDR amino acid sequences as found in a particular human antibody exhibiting immunospecificity for the extracellular portion of ZIP12, or a variant or fragment thereof. An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids. Such a human antibody maintains its function of selectively binding to the extracellular portion of ZIP12 or a variant or fragment thereof.

The term “human monoclonal antibody” can include a monoclonal antibody with substantially or entirely human CDR amino acid sequences produced, for example by recombinant methods such as production by a phage library, by lymphocytes or by hybridoma cells.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (by so-called “affinity maturation”), to humanize the antibody, to improve its production in cell culture, and/or to reduce its immunogenicity in a subject.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen, for example, the extracellular portion of ZIP12, or a variant or fragment thereof. Preferably, the fragment is an epitope. The antigen-binding region may be a hypervariable CDR or a functional portion thereof. The term “functional portion” of a CDR can mean a sequence within the CDR which shows specific affinity for the target antigen. The functional portion of a CDR may comprise a ligand which specifically binds to the extracellular portion of ZIP12, or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The amino acid sequence boundaries of a CDR can be determined by using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme).

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can mean a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about no to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains-and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antigen-binding fragment thereof, with respect to the first aspect of the invention, is immunospecific for an epitope within the extracellular portion of ZIP12. The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may be a single domain antibody (sdAb), otherwise referred to as a nanobody, which the skilled person would understand is an antibody fragment consisting of a single monomeric variable antibody domain.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F (ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region of the antibody or antigen-binding fragment thereof is capable of immunoreacting with the extracellular portion of ZIP12, or a variant or fragment thereof, by specifically binding therewith. The antibody or antigen-binding fragment thereof can preferably selectively interact with an antigen (extracellular portion of ZIP12) with an affinity constant of approximately to 10⁻⁵ to 10⁻¹³ M⁻¹, preferably to 10⁻⁶ to 10⁻⁹ M⁻¹, even more preferably, to 10⁻¹⁰ to 10₋₁₂ M⁻¹. The antibody or antigen-binding fragment thereof preferably does not substantially bind to ZIP4 and/or ZIP13, such that the affinity constant is approximately more than to 10⁻¹⁰ M⁻¹, 10⁻⁹ M⁻¹, 10⁻⁸ M⁻¹, 10⁻⁷ M⁻¹, or to 10⁻⁶ M⁻¹, preferably more than 10⁻⁵M⁻¹, 10⁻⁴M⁻¹ or to 10⁻³M⁻¹ and even more preferably to 10⁻² M⁻¹ to 10⁻¹ M⁻¹ or 10⁻² M⁻¹ and most preferably 10⁺¹M⁻¹, 10⁺²M⁻¹ or 10⁺³M⁻¹.

The term “immunoreact” can mean the binding region is capable of eliciting an immune response upon binding with the extracellular region of ZIP12, or an epitope thereof.

The term “epitope” can mean any region of an antigen with the ability to elicit, and combine with, a binding region of the antibody or antigen-binding fragment thereof.

Thus, the antibody the antibody or antigen-binding fragment thereof may comprise a heavy chain. The heavy chain may be selected from the group consisting of IgA; IgD; IgE; IgG and IgM. Preferably, the heavy chain is an IgG. Preferably, the heavy chain is an IgA.

The heavy chain may be an IgG1. The heavy chain may be an IgG2. The heavy chain may be an IgG3. The heavy chain may be an IgG4. The heavy chain may be an IgA1. The heavy chain may be an IgA2.

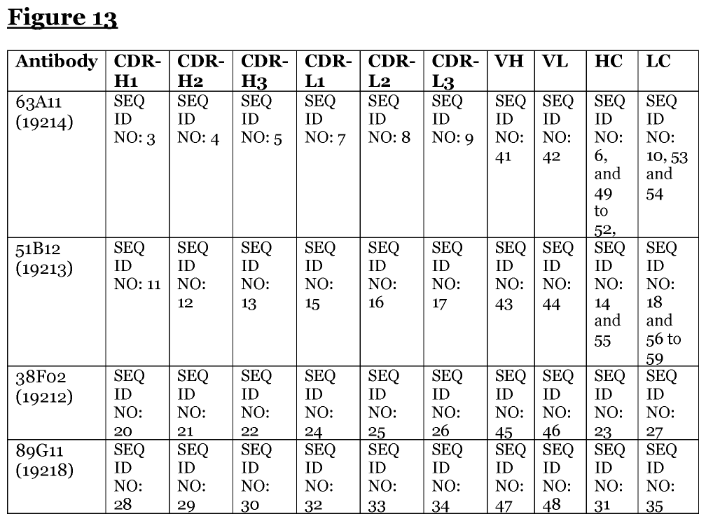

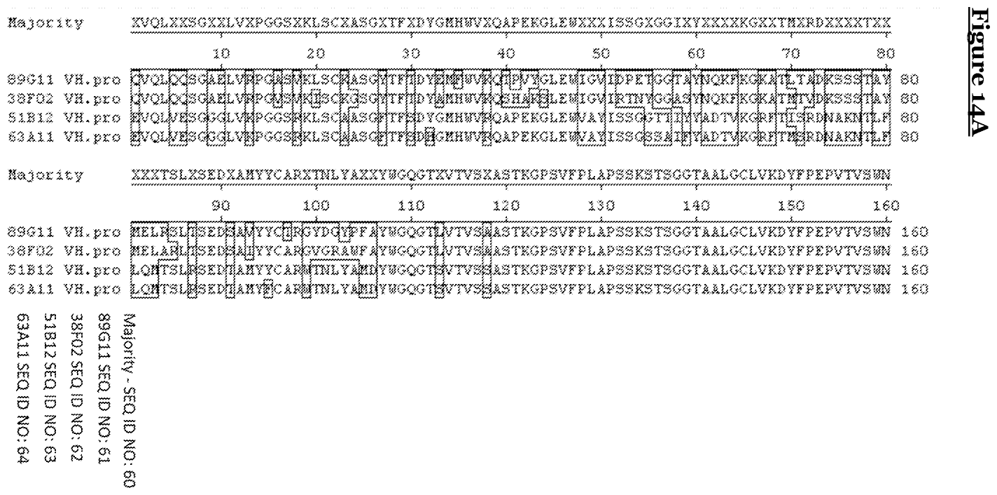

As described in the Examples and as shown in FIG. 2, the inventors have surprisingly demonstrated that the antibodies referred to herein as 19212 (“38Fo2”), 19213 (“51B12”), 19214 (“63A11”) and 19218 (“89G11”) are all able to significantly target and inhibit the function of ZIP12. The CDR/VH/VL, HC and LC sequences of these four antibodies are conveniently summarised in the table shown in FIG. 13. Advantageously, and preferably, the inventors have also demonstrated that both the parent and humanised antibodies of 63A11 and 51B12 lineage retain activity and demonstrate no cross-reactivity with ZIP4.

19214 (“63A11”)

Accordingly, in one embodiment, the antibody or antigen-binding fragment thereof is referred to herein as 63A11. The antibody or antigen-binding fragment thereof may comprise a CDR-H1 domain of SEQ ID No: 3, which is provided herein, as follows:

[SEQ ID No: 3]
DHGMH

Thus, preferably, the antibody or antigen-binding fragment thereof comprises a CDR-H1 domain comprising or consisting of a sequence as substantially set out in SEQ ID No: 3, or a variant or fragment thereof.

The antibody or antigen-binding fragment thereof may comprise a CDR-H2 domain of SEQ ID No: 4, which is provided herein, as follows:

[SEQ ID No: 4]
YISSGSSAIFYADTVKG

Thus, preferably the antibody or antigen-binding fragment thereof comprises a CDR-H2 domain comprising or consisting of a sequence as substantially set out in SEQ ID No: 4, or a variant or fragment thereof.

The antibody or antigen-binding fragment thereof may comprise a CDR-H3 domain of SEQ ID No: 5, which is provided herein, as follows:

[SEQ ID No: 5]
WTNLYAMDY

Thus, preferably the antibody or antigen-binding fragment thereof comprises a CDR-H3 domain comprising or consisting of a sequence as substantially set out in SEQ ID No: 5, or a variant or fragment thereof.

Preferably, the antibody or antigen-binding fragment thereof comprises a CDR-H1 domain comprising or consisting of SEQ ID No: 3, a CDR-H2 domain comprising or consisting of SEQ ID No: 4 and/or a CDR-H3 domain comprising or consisting of SEQ ID No: 5. Preferably, however, the antibody or antigen-binding fragment thereof comprises a CDR-H1 domain comprising or consisting of SEQ ID No: 3, a CDR-H2 domain comprising or consisting of SEQ ID No: 4 and a CDR-H3 domain comprising or consisting of SEQ ID No: 5.

The antibody or antigen-binding fragment thereof may comprise a heavy chain variable (VH) sequence as set out in SEQ ID No: 41, which is provided herein, as follows:

[SEQ ID No: 41]
EVQLVESGGGLVKPGGSRKLSCAASGFTFSDHGMHWVRQAPEKGLEWVA
YISSGSSAIFYADTVKGRFTMSRDNAKNTLFLQMTSLRSEDTAMYFCAR
WTNLYAMDYWGQGTSVTVSS

Preferably, the antibody or antigen-binding fragment thereof comprises a heavy chain variable (VH) region comprising or consisting of a sequence as substantially set out in SEQ ID No: 41, or a variant or fragment thereof.

The antibody or antigen-binding fragment thereof may comprise a heavy chain sequence as set out in SEQ ID No: 6, which is provided herein, as follows:

[SEQ ID No: 6]
EVQLVESGGGLVKPGGSRKLSCAASGFTFSDHGMHWVRQAPEKGLEWVA
YISSGSSAIFYADTVKGRFTMSRDNAKNTLFLQMTSLRSEDTAMYFCAR
WTNLYAMDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Preferably, the antibody or antigen-binding fragment thereof comprises a heavy chain region comprising or consisting of a sequence as substantially set out in SEQ ID No: 6, or a variant or fragment thereof.

The antibody or antigen-binding fragment thereof may comprise a heavy chain sequence as set out in SEQ ID No: 49, which is provided herein, as follows:

[SEQ ID No: 49]
QVQLVESGGGVVQPGRSLRLSCAASGFTFSDHGMHWVRQAPGKGLEWVAYISSGSSAIFYADTVKGRFTI
SRDNSKNTLYLQMNSLRAEDTAVYYCARWTNLYAMDYWGQGTTVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK

Preferably, the antibody or antigen-binding fragment thereof comprises a heavy chain region comprising or consisting of a sequence as substantially set out in SEQ ID No: 49, or a variant or fragment thereof.

The antibody or antigen-binding fragment thereof may comprise a heavy chain sequence as set out in SEQ ID No: 50, which is provided herein, as follows:

[SEQ ID No: 50]
QVQLVESGGGVVQPGRSLRLSCAASGFTFSDHGMHWVRQAPGKGLEWVAYISSGSSAIFYADTVKGRFTI
SRDNSKNTLYLQMNSLRAEDTAVYFCARWTNLYAMDYWGQGTTVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK

Preferably, the antibody or antigen-binding fragment thereof comprises a heavy chain region comprising or consisting of a sequence as substantially set out in SEQ ID No: 50, or a variant or fragment thereof.

The antibody or antigen-binding fragment thereof may comprise a heavy chain sequence as set out in SEQ ID No: 51, which is provided herein, as follows:

[SEQ ID No: 51]
QVQLVESGGGVVQPGRSLRLSCAASGFTFSDHGMHWVRQAPGKGLEWVAYISSGSSAIFYADTVKGRFTM
SRDNSKNTLYLQMNSLRAEDTAVYYCARWTNLYAMDYWGQGTTVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK

Preferably, the antibody or antigen-binding fragment thereof comprises a heavy chain region comprising or consisting of a sequence as substantially set out in SEQ ID No: 51, or a variant or fragment thereof.

The antibody or antigen-binding fragment thereof may comprise a heavy chain sequence as set out in SEQ ID No: 52, which is provided herein, as follows:

[SEQ ID No: 52]
QVQLVESGGGVVQPGRSLRLSCAASGFTFSDHGMHWVRQAPGKGLEWVAYISSGSSAIFYADTVKGRFTM
SRDNSKNTLYLQMNSLRAEDTAVYFCARWTNLYAMDYWGQGTTVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK

Preferably, the antibody or antigen-binding fragment thereof comprises a heavy chain region comprising or consisting of a sequence as substantially set out in SEQ ID No: 52, or a variant or fragment thereof.

The antibody or antigen-binding fragment thereof may comprise a light chain CDR-L1 domain of SEQ ID No: 7, which is provided herein, as follows:

[SEQ ID No: 7]
RASKSVSTSGYSYMH

Thus, preferably the antibody or antigen-binding fragment thereof comprises a CDR-L1 domain comprising or consisting of a sequence as substantially set out in SEQ ID No: 7, or a variant or fragment thereof.

The antibody or antigen-binding fragment thereof may comprise a CDR-L2 domain of SEQ ID No: 8, which is provided herein, as follows:

[SEQ ID No: 8]
LASNLES

Thus, preferably the antibody or antigen-binding fragment thereof comprises a CDR-L2 domain comprising or consisting of a sequence as substantially set out in SEQ ID No: 8, or a variant or fragment thereof.

The antibody or antigen-binding fragment thereof may comprise a CDR-L3 domain of SEQ ID No: 9, which is provided herein, as follows:

[SEQ ID No: 9]
QHSRELPLT

Thus, preferably the antibody or antigen-binding fragment thereof comprises a CDR-L3 domain comprising or consisting of a sequence as substantially set out in SEQ ID No: 9, or a variant or fragment thereof.

Preferably, the antibody or antigen-binding fragment thereof comprises a CDR-L1 domain comprising or consisting of SEQ ID No: 7, a CDR-L2 domain comprising or consisting of SEQ ID No: 8, and/or a CDR-L3 domain comprising or consisting of SEQ ID No: 9. However, preferably the antibody or antigen-binding fragment thereof comprises a CDR-L1 domain comprising or consisting of SEQ ID No: 7, a CDR-L2 domain comprising or consisting of SEQ ID No: 8, and a CDR-L3 domain comprising or consisting of SEQ ID No: 9.

The antibody or antigen-binding fragment thereof may comprise a light chain variable (VL) sequence as set out in SEQ ID No: 42, which is provided herein, as follows:

[SEQ ID No: 42]
DIVLTQSPASLAVSLGQRATISCRASKSVSTSGYSYMHWYQQKPGQPPK
LLIYLASNLESGVPARFSGSGSGTDFTLNIHPVEEEDAATYYCQHSREL
PLTFGAGTKLELK

Preferably, the antibody or antigen-binding fragment thereof comprises a light chain variable region comprising or consisting of a sequence as substantially set out in SEQ ID No: 42, or a variant or fragment thereof.

The antibody or antigen-binding fragment thereof may comprise a light chain sequence as set out in SEQ ID No: 10, which is provided herein, as follows:

[SEQ ID No: 10]
DIVLTQSPASLAVSLGQRATISCRASKSVSTSGYSYMHWYQQKPGQPPK
LLIYLASNLESGVPARFSGSGSGTDFTLNIHPVEEEDAATYYCQHSREL
PLTFGAGTKLELKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 10, or a variant or fragment thereof.

The antibody or antigen-binding fragment thereof may comprise a light chain sequence as set out in SEQ ID No: 53, which is provided herein, as follows:

[SEQ ID No: 53]
DIVMTQSPDSLAVSLGERATINCRASKSVSTSGYSYMHWYQQKPGQPPK
LLIYLASNLESGVPDRFSGSGSGTDFTLTISSLQAEDVAVYYCQHSREL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 53, or a variant or fragment thereof.

The antibody or antigen-binding fragment thereof may comprise a light chain sequence as set out in SEQ ID No: 54, which is provided herein, as follows:

[SEQ ID No: 54]
DIVLTQSPDSLAVSLGERATINCRASKSVSTSGYSYMHWYQQKPGQPPK
LLIYLASNLESGVPDRFSGSGSGTDFTLTISSLQAEDVAVYYCQHSREL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 54, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 3, a CDR-H2 domain comprising or consisting of SEQ ID No: 4; a CDR-H3 domain comprising or consisting of SEQ ID No: 5, a CDR-L1 domain comprising or consisting of SEQ ID No: 7, a CDR-L2 domain comprising or consisting of SEQ ID No: 8, and a CDR-L3 domain comprising or consisting of SEQ ID No: 9.

Preferably, the antibody or antigen-binding fragment thereof comprises a heavy chain variable region comprising or consisting of SEQ ID No: 41 and a light chain variable region comprising or consisting of SEQ ID No: 42.

Preferably, the antibody or antigen-binding fragment thereof comprises a heavy chain region comprising or consisting of SEQ ID No: 6 and a light chain region comprising or consisting of SEQ ID No: 10.

Preferably, the antibody or antigen-binding fragment thereof comprises a heavy chain region selected from a group comprising or consisting of: SEQ ID No: 49, SEQ ID No: 50, SEQ ID No: 51 and SEQ ID No: 52 and a light chain region comprising or consisting of SEQ ID No: 53 or SEQ ID No: 54.

Preferably, the antibody or antigen-binding fragment thereof comprises a heavy chain region comprising or consisting of: SEQ ID No: 49 and a light chain region comprising or consisting of SEQ ID No: 53 or SEQ ID No: 54. Preferably, the antibody or antigen-binding fragment thereof comprises a heavy chain region comprising or consisting of: SEQ ID No: 50 and a light chain region comprising or consisting of SEQ ID No: 53 or SEQ ID No: 54. Preferably, the antibody or antigen-binding fragment thereof comprises a heavy chain comprising or consisting of: SEQ ID No: 51 and a light chain region comprising or consisting of SEQ ID No: 53 or SEQ ID No: 54. Preferably, the antibody or antigen-binding fragment thereof comprises a heavy chain region consisting of: SEQ ID No: 52 and a light chain region comprising or consisting of SEQ ID No: 53 or SEQ ID No: 54.

19213 (“51B12”)

In one embodiment, the antibody or antigen-binding fragment thereof is referred to herein as 51B12. The antibody or antigen-binding fragment thereof may comprise a CDR-H1 domain of SEQ ID No: 11, which is provided herein, as follows:

[SEQ ID No: 11]
DYGMH

Thus, preferably, the antibody or antigen-binding fragment thereof comprises a CDR-H1 domain comprising or consisting of a sequence as substantially set out in SEQ ID No: 11, or a variant or fragment thereof.

The antibody or antigen-binding fragment thereof may comprise a CDR-H2 domain of SEQ ID No: 12, which is provided herein, as follows:

[SEQ ID No: 12]
YISSGGTTIYYADTVKG

Thus, preferably the antibody or antigen-binding fragment thereof comprises a CDR-H2 domain comprising or consisting of a sequence as substantially set out in SEQ ID No: 12, or a variant or fragment thereof.

The antibody or antigen-binding fragment thereof may comprise a CDR-H3 domain of SEQ ID No: 13, which is provided herein, as follows:

[SEQ ID No: 13]
WTNLYAMDY

Thus, preferably the antibody or antigen-binding fragment thereof comprises a CDR-H3 domain comprising or consisting of a sequence as substantially set out in SEQ ID No: 13, or a variant or fragment thereof.

Preferably, the antibody or antigen-binding fragment thereof comprises a CDR-H1 domain comprising or consisting of SEQ ID No: ii, a CDR-H2 domain comprising or consisting of SEQ ID No: 12 and/or a CDR-H3 domain comprising or consisting of SEQ ID No: 13. Preferably, however, the antibody or antigen-binding fragment thereof comprises a CDR-H1 domain comprising or consisting of SEQ ID No: ii, a CDR-H2 domain comprising or consisting of SEQ ID No: 12 and a CDR-H3 domain comprising or consisting of SEQ ID No: 13.

The antibody or antigen-binding fragment thereof may comprise a heavy chain variable (VH) sequence as set out in SEQ ID No: 43, which is provided herein, as follows:

[SEQ ID No: 43]
EVQLVESGGGLVKPGGSRKLSCAASGFTFSDYGMHWVRQAPEKGLEWVA
YISSGGTTIYYADTVKGRFTISRDNAKNTLFLQMTSLRSEDTAMYYCAR
WTNLYAMDYWGQGTSVTVSS

Preferably, the antibody or antigen-binding fragment thereof comprises a heavy chain variable (VH) region comprising or consisting of a sequence as substantially set out in SEQ ID No: 43, or a variant or fragment thereof.

The antibody or antigen-binding fragment thereof may comprise a heavy chain sequence as set out in SEQ ID No: 14, which is provided herein, as follows:

[SEQ ID No: 14]
EVQLVESGGGLVKPGGSRKLSCAASGFTFSDYGMHWVRQAPEKGLEWVA
YISSGGTTIYYADTVKGRFTISRDNAKNTLFLQMTSLRSEDTAMYYCAR
WTNLYAMDYWGQGTSVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Preferably, the antibody or antigen-binding fragment thereof comprises a heavy chain region comprising or consisting of a sequence as substantially set out in SEQ ID No: 14, or a variant or fragment thereof.

The antibody or antigen-binding fragment thereof may comprise a heavy chain sequence as set out in SEQ ID No: 55, which is provided herein, as follows:

[SEQ ID No: 55]
QVQLVESGGGVVQPGRSLRLSCAASGFTFSDYGMHWVRQAPGKGLEWVA
YISSGGTTIYYADTVKGRFTISRDNSKNTLYLQMNSLRAEDTAVYYCAR
WTNLYAMDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Preferably, the antibody or antigen-binding fragment thereof comprises a heavy chain region comprising or consisting of a sequence as substantially set out in SEQ ID No: 55, or a variant or fragment thereof.

The antibody or antigen-binding fragment thereof may comprise a light chain CDR-L1 domain of SEQ ID No: 15, which is provided herein, as follows:

[SEQ ID No: 15]
RASKSVSTSGYSYMH

Thus, preferably the antibody or antigen-binding fragment thereof comprises a CDR-L1 domain comprising or consisting of a sequence as substantially set out in SEQ ID No: 15, or a variant or fragment thereof.

The antibody or antigen-binding fragment thereof may comprise a CDR-L2 domain of SEQ ID No: 16, which is provided herein, as follows:

[SEQ ID No: 16]
LASNLES

Thus, preferably the antibody or antigen-binding fragment thereof comprises a CDR-L2 domain comprising or consisting of a sequence as substantially set out in SEQ ID No: 16, or a variant or fragment thereof.

The antibody or antigen-binding fragment thereof may comprise a CDR-L3 domain of SEQ ID No: 17, which is provided herein, as follows:

[SEQ ID No: 17]
QHSREVPYT

Thus, preferably the antibody or antigen-binding fragment thereof comprises a CDR-L3 domain comprising or consisting of a sequence as substantially set out in SEQ ID No: 17, or a variant or fragment thereof.

Preferably, the antibody or antigen-binding fragment thereof comprises a CDR-L1 domain comprising or consisting of SEQ ID No: 15, a CDR-L2 domain comprising or consisting of SEQ ID No: 16, and/or a CDR-L3 domain comprising or consisting of SEQ ID No: 17. However, preferably the antibody or antigen-binding fragment thereof comprises a CDR-L1 domain comprising or consisting of SEQ ID No: 15, a CDR-L2 domain comprising or consisting of SEQ ID No: 16, and a CDR-L3 domain comprising or consisting of SEQ ID No: 17.

The antibody or antigen-binding fragment thereof may comprise a light chain variable (VL) sequence as set out in SEQ ID No: 44, which is provided herein, as follows:

[SEQ ID No: 44]
DIVLTQSPASLAVSLGQRATISCRASKSVSTSGYSYMHWYQQKPGQPPK
FLIYLASNLESGVPARFSGSGSGTDFTLNIHPVEEEDAATYYCQHSREV
PYTFGGGTKLEIK

Preferably, the antibody or antigen-binding fragment thereof comprises a light chain variable (VL) region comprising or consisting of a sequence as substantially set out in SEQ ID No: 44, or a variant or fragment thereof.

The antibody or antigen-binding fragment thereof may comprise a light chain sequence as set out in SEQ ID No: 18, which is provided herein, as follows:

[SEQ ID No: 18]
DIVLTQSPASLAVSLGQRATISCRASKSVSTSGYSYMHWYQQKPGQPPK
FLIYLASNLESGVPARFSGSGSGTDFTLNIHPVEEEDAATYYCQHSREV
PYTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 18, or a variant or fragment thereof.

The antibody or antigen-binding fragment thereof may comprise a light chain sequence as set out in SEQ ID No: 56, which is provided herein, as follows:

[SEQ ID No: 56]
DIVMTQSPDSLAVSLGERATINCRASKSVSTSGYSYMHWYQQKPGQPPK
LLIYLASNLESGVPDRFSGSGSGTDFTLTISSLQAEDVAVYYCQHSREV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 56, or a variant or fragment thereof.

The antibody or antigen-binding fragment thereof may comprise a light chain sequence as set out in SEQ ID No: 57, which is provided herein, as follows:

[SEQ ID No: 57]
DIVLTQSPDSLAVSLGERATINCRASKSVSTSGYSYMHWYQQKPGQPPK
LLIYLASNLESGVPDRFSGSGSGTDFTLTISSLQAEDVAVYYCQHSREV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 57, or a variant or fragment thereof.

The antibody or antigen-binding fragment thereof may comprise a light chain sequence as set out in SEQ ID No: 58, which is provided herein, as follows:

[SEQ ID No: 58]
DIVMTQSPDSLAVSLGERATINCRASKSVSTSGYSYMHWYQQKPGQPPK
FLIYLASNLESGVPDRFSGSGSGTDFTLTISSLQAEDVAVYYCQHSREV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 58, or a variant or fragment thereof.

The antibody or antigen-binding fragment thereof may comprise a light chain sequence as set out in SEQ ID No: 59, which is provided herein, as follows:

[SEQ ID No: 59]
DIVLTQSPDSLAVSLGERATINCRASKSVSTSGYSYMHWYQQKPGQPPK
FLIYLASNLESGVPDRFSGSGSGTDFTLTISSLQAEDVAVYYCQHSREV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 59, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 11, a CDR-H2 domain comprising or consisting of SEQ ID No: 12; a CDR-H3 domain comprising or consisting of SEQ ID No: 13, a CDR-L1 domain comprising or consisting of SEQ ID No: 15, a CDR-L2 domain comprising or consisting of SEQ ID No: 16, and a CDR-L3 domain comprising or consisting of SEQ ID No: 17.

Preferably, the antibody or antigen-binding fragment thereof comprises a heavy chain variable region comprising or consisting of SEQ ID No: 43 and a light chain variable region comprising or consisting of SEQ ID No: 44.

Preferably, the antibody or antigen-binding fragment thereof comprises a heavy chain region comprising or consisting of SEQ ID No: 14 and a light chain region comprising or consisting of SEQ ID No: 18.

Preferably, the antibody or antigen-binding fragment thereof comprises a heavy chain region comprising or consisting of SEQ ID No: 55 and a light chain region selected from a group comprising or consisting of: SEQ ID No: 56, SEQ ID No: 57, SEQ ID No: 58 and SEQ ID No: 59.

Preferably, the antibody or antigen-binding fragment thereof comprises a heavy chain region comprising or consisting of SEQ ID No: 55 and a light chain region comprising or consisting of SEQ ID No: 56. Preferably, the antibody or antigen-binding fragment thereof comprises a heavy chain region comprising or consisting of SEQ ID No: 55 and a light chain region comprising or consisting of SEQ ID No: 57. Preferably, the antibody or antigen-binding fragment thereof comprises a heavy chain region comprising or consisting of SEQ ID No: 55 and a light chain region comprising or consisting of SEQ ID No: 58. Preferably, the antibody or antigen-binding fragment thereof comprises a heavy chain region comprising or consisting of SEQ ID No: 55 and a light chain region comprising or consisting of SEQ ID No: 59.

38F02 (19212)

In one embodiment, the antibody or antigen-binding fragment thereof is referred to herein as 19212. The antibody or antigen-binding fragment thereof may comprise a CDR-H1 domain of SEQ ID No: 20, which is provided herein, as follows:

[SEQ ID No: 20]
DYAMH

Thus, preferably, the antibody or antigen-binding fragment thereof comprises a CDR-H1 domain comprising or consisting of a sequence as substantially set out in SEQ ID No: 20, or a variant or fragment thereof.

The antibody or antigen-binding fragment thereof may comprise a CDR-H2 domain of SEQ ID No: 21, which is provided herein, as follows:

[SEQ ID No: 21]
VIRTNYGGASYNQKFKG

Thus, preferably the antibody or antigen-binding fragment thereof comprises a CDR-H2 domain comprising or consisting of a sequence as substantially set out in SEQ ID No: 21, or a variant or fragment thereof.

The antibody or antigen-binding fragment thereof may comprise a CDR-H3 domain of SEQ ID No: 22, which is provided herein, as follows:

[SEQ ID No: 22]
GVGRAWFAY

Thus, preferably the antibody or antigen-binding fragment thereof comprises a CDR-H3 domain comprising or consisting of a sequence as substantially set out in SEQ ID No: 22, or a variant or fragment thereof.

Preferably, the antibody or antigen-binding fragment thereof comprises a CDR-H1 domain comprising or consisting of SEQ ID No: 20, a CDR-H2 domain comprising or consisting of SEQ ID No: 21 and/or a CDR-H3 domain comprising or consisting of SEQ ID No: 22. Preferably, however, the antibody or antigen-binding fragment thereof comprises a CDR-H1 domain comprising or consisting of SEQ ID No: 20, a CDR-H2 domain comprising or consisting of SEQ ID No: 21 and a CDR-H3 domain comprising or consisting of SEQ ID No: 22.

The antibody or antigen-binding fragment thereof may comprise a heavy chain variable (VH) sequence as set out in SEQ ID No: 45, which is provided herein, as follows:

[SEQ ID No: 45]
QVQLQQSGAELVRPGVSVKISCKGSGYTFTDYAMHWVKQSHAKSLEWIG
VIRTNYGGASYNQKFKGKATMTVDKSSSTAYMELARLTSEDSAIYYCAR
GVGRAWFAYWGQGTLVTVSA

Preferably, the antibody or antigen-binding fragment thereof comprises a heavy chain variable (VH) region comprising or consisting of a sequence as substantially set out in SEQ ID No: 45, or a variant or fragment thereof.

The antibody or antigen-binding fragment thereof may comprise a heavy chain sequence as set out in SEQ ID No: 23, which is provided herein, as follows:

[SEQ ID No: 23]
QVQLQQSGAELVRPGVSVKISCKGSGYTFTDYAMHWVKQSHAKSLEWIG
VIRTNYGGASYNQKFKGKATMTVDKSSSTAYMELARLTSEDSAIYYCAR
GVGRAWFAYWGQGTLVTVSAASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Preferably, the antibody or antigen-binding fragment thereof comprises a heavy chain region comprising or consisting of a sequence as substantially set out in SEQ ID No: 23, or a variant or fragment thereof.

The antibody or antigen-binding fragment thereof may comprise a light chain CDR-L1 domain of SEQ ID No: 24, which is provided herein, as follows:

[SEQ ID No: 24]
KASQSVDYDGDSYMN

Thus, preferably the antibody or antigen-binding fragment thereof comprises a CDR-L1 domain comprising or consisting of a sequence as substantially set out in SEQ ID No: 24, or a variant or fragment thereof.

The antibody or antigen-binding fragment thereof may comprise a CDR-L2 domain of SEQ ID No: 25, which is provided herein, as follows:

[SEQ ID No: 25]
AASNLES

Thus, preferably the antibody or antigen-binding fragment thereof comprises a CDR-L2 domain comprising or consisting of a sequence as substantially set out in SEQ ID No: 25, or a variant or fragment thereof.

The antibody or antigen-binding fragment thereof may comprise a CDR-L3 domain of SEQ ID No: 26, which is provided herein, as follows:

[SEQ ID No: 26]
QQSYDDPYT

Thus, preferably the antibody or antigen-binding fragment thereof comprises a CDR-L3 domain comprising or consisting of a sequence as substantially set out in SEQ ID No: 26, or a variant or fragment thereof.

Preferably, the antibody or antigen-binding fragment thereof comprises a CDR-L1 domain comprising or consisting of SEQ ID No: 24, a CDR-L2 domain comprising or consisting of SEQ ID No: 25, and/or a CDR-L3 domain comprising or consisting of SEQ ID No: 26. However, preferably the antibody or antigen-binding fragment thereof comprises a CDR-L1 domain comprising or consisting of SEQ ID No: 24, a CDR-L2 domain comprising or consisting of SEQ ID No: 25, and a CDR-L3 domain comprising or consisting of SEQ ID No: 26.

The antibody or antigen-binding fragment thereof may comprise a light chain variable (VL) sequence as set out in SEQ ID No: 46, which is provided herein, as follows:

[SEQ ID No: 46]
DIVLTQSPASLAVSLGQRATISCKASQSVDYDGDSYMNWYQQKPGQPPK
LLIYAASNLESGIPARFSGSGSGTDFTLNIHPVEEEDAATYYCQQSYDD
PYTFGGGTKLEIK

Preferably, the antibody or antigen-binding fragment thereof comprises a light chain variable (VL) region comprising or consisting of a sequence as substantially set out in SEQ ID No: 46, or a variant or fragment thereof.

The antibody or antigen-binding fragment thereof may comprise a light chain sequence as set out in SEQ ID No: 27, which is provided herein, as follows:

[SEQ ID No: 27]
DIVLTQSPASLAVSLGQRATISCKASQSVDYDGDSYMNWYQQKPGQPPK
LLIYAASNLESGIPARFSGSGSGTDFTLNIHPVEEEDAATYYCQQSYDD
PYTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

Preferably, the antibody or antigen-binding fragment thereof comprises a light chain region comprising or consisting of a sequence as substantially set out in SEQ ID No: 27, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 20, a CDR-H2 domain comprising or consisting of SEQ ID No: 21; a CDR-H3 domain comprising or consisting of SEQ ID No: 22, a CDR-L1 domain comprising or consisting of SEQ ID No: 24, a CDR-L2 domain comprising or consisting of SEQ ID No: 25, and a CDR-L3 domain comprising or consisting of SEQ ID No: 26.

Preferably, the antibody or antigen-binding fragment thereof comprises a heavy chain variable region comprising or consisting of SEQ ID No: 45 and a light chain variable region comprising or consisting of SEQ ID No: 46.

Preferably, the antibody or antigen-binding fragment thereof comprises a heavy chain region comprising or consisting of SEQ ID No: 23 and a light chain region comprising or consisting of SEQ ID No: 27.

89G11(19218)

In one embodiment, the antibody or antigen-binding fragment thereof is referred to herein as 19218. The antibody or antigen-binding fragment thereof may comprise a CDR-H1 domain of SEQ ID No: 28, which is provided herein, as follows:

[SEQ ID No: 28]
DYEMF

Thus, preferably, the antibody or antigen-binding fragment thereof comprises a CDR-H1 domain comprising or consisting of a sequence as substantially set out in SEQ ID No: 28, or a variant or fragment thereof.

The antibody or antigen-binding fragment thereof may comprise a CDR-H2 domain of SEQ ID No: 29, which is provided herein, as follows:

[SEQ ID No: 29]
VIDPETGGTAYNQKFKG

Thus, preferably the antibody or antigen-binding fragment thereof comprises a CDR-H2 domain comprising or consisting of a sequence as substantially set out in SEQ ID No: 29, or a variant or fragment thereof.

The antibody or antigen-binding fragment thereof may comprise a CDR-H3 domain of SEQ ID No: 30, which is provided herein, as follows:

[SEQ ID No: 30]
GYGDYPFAY

Thus, preferably the antibody or antigen-binding fragment thereof comprises a CDR-H3 domain comprising or consisting of a sequence as substantially set out in SEQ ID No: 30, or a variant or fragment thereof.

Preferably, the antibody or antigen-binding fragment thereof comprises a CDR-H1 domain comprising or consisting of SEQ ID No: 28, a CDR-H2 domain comprising or consisting of SEQ ID No: 29 and/or a CDR-H3 domain comprising or consisting of SEQ ID No: 30. Preferably, however, the antibody or antigen-binding fragment thereof comprises a CDR-H1 domain comprising or consisting of SEQ ID No: 28, a CDR-H2 domain comprising or consisting of SEQ ID No: 29 and a CDR-H3 domain comprising or consisting of SEQ ID No: 30.

The antibody or antigen-binding fragment thereof may comprise a heavy chain (VH) variable sequence as set out in SEQ ID No: 47, which is provided herein, as follows:

[SEQ ID No: 47]
QVQLQQSGAELVRPGASVKLSCKASGYTFTDYEMFWVKQTPVYGLEWIG
VIDPETGGTAYNQKFKGKATLTADKSSSTAYMELRSLTSEDSAVYYCTR
GYDGYPFAYWGQGTLVTVSA

Preferably, the antibody or antigen-binding fragment thereof comprises a heavy chain variable (VH) region comprising or consisting of a sequence as substantially set out in SEQ ID No: 47, or a variant or fragment thereof.

The antibody or antigen-binding fragment thereof may comprise a heavy chain sequence as set out in SEQ ID No: 31, which is provided herein, as follows:

[SEQ ID No: 31]
QVQLQQSGAELVRPGASVKLSCKASGYTFTDYEMFWVKQTPVYGLEWIG
VIDPETGGTAYNQKFKGKATLTADKSSSTAYMELRSLTSEDSAVYYCTR
GYDGYPFAYWGQGTLVTVSAASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Preferably, the antibody or antigen-binding fragment thereof comprises a heavy chain region comprising or consisting of a sequence as substantially set out in SEQ ID No: 31, or a variant or fragment thereof.

The antibody or antigen-binding fragment thereof may comprise a light chain CDR-L1 domain of SEQ ID No: 32, which is provided herein, as follows:

[SEQ ID No: 32]
KSSQLYSSNQKNFLA

Thus, preferably the antibody or antigen-binding fragment thereof comprises a CDR-L1 domain comprising or consisting of a sequence as substantially set out in SEQ ID No: 32, or a variant or fragment thereof.

The antibody or antigen-binding fragment thereof may comprise a CDR-L2 domain of SEQ ID No: 33, which is provided herein, as follows:

[SEQ ID No: 33]
WASTRES

Thus, preferably the antibody or antigen-binding fragment thereof comprises a CDR-L2 domain comprising or consisting of a sequence as substantially set out in SEQ ID No: 33, or a variant or fragment thereof.

The antibody or antigen-binding fragment thereof may comprise a CDR-L3 domain of SEQ ID No: 34, which is provided herein, as follows:

[SEQ ID No: 34]
LQYLSSYT

Thus, preferably the antibody or antigen-binding fragment thereof comprises a CDR-L3 domain comprising or consisting of a sequence as substantially set out in SEQ ID No: 34, or a variant or fragment thereof.

Preferably, the antibody or antigen-binding fragment thereof comprises a CDR-L1 domain comprising or consisting of SEQ ID No: 32, a CDR-L2 domain comprising or consisting of SEQ ID No: 33, and/or a CDR-L3 domain comprising or consisting of SEQ ID No: 34. However, preferably the antibody or antigen-binding fragment thereof comprises a CDR-L1 domain comprising or consisting of SEQ ID No: 32, a CDR-L2 domain comprising or consisting of SEQ ID No: 33, and a CDR-L3 domain comprising or consisting of SEQ ID No: 34.

The antibody or antigen-binding fragment thereof may comprise a light chain variable (VL) sequence as set out in SEQ ID No: 48, which is provided herein, as follows:

[SEQ ID No: 48]
NIMMTQSPSSLAVSAGEKVTMSCKSSQSVLYSSNQKNFLAWYQQKPGQS
PKLLIYWASTRESGVPDRFTGSGSGTDFTLTISSVRAEDLAVYYCLQYL
SSYTFGGGTRLDIK

Preferably, the antibody or antigen-binding fragment thereof comprises a light chain variable (VL) region comprising or consisting of a sequence as substantially set out in SEQ ID No: 48, or a variant or fragment thereof.

The antibody or antigen-binding fragment thereof may comprise a light chain sequence as set out in SEQ ID No: 35, which is provided herein, as follows:

[SEQ ID No: 35]
NIMMTQSPSSLAVSAGEKVTMSCKSSQSVLYSSNQKNFLAWYQQKPGQS
PKLLIYWASTRESGVPDRFTGSGSGTDFTLTISSVRAEDLAVYYCLQYL
SSYTFGGGTRLDIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

Preferably, the antibody or antigen-binding fragment thereof comprises a light chain region comprising or consisting of a sequence as substantially set out in SEQ ID No: 35, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 28, a CDR-H2 domain comprising or consisting of SEQ ID No: 29; a CDR-H3 domain comprising or consisting of SEQ ID No: 30, a CDR-L1 domain comprising or consisting of SEQ ID No: 32, a CDR-L2 domain comprising or consisting of SEQ ID No: 33, and a CDR-L3 domain comprising or consisting of SEQ ID No: 34.

Preferably, the antibody or antigen-binding fragment thereof comprises a heavy chain variable region comprising or consisting of SEQ ID No: 47 and a light chain variable region comprising or consisting of SEQ ID No: 48.

Preferably, the antibody or antigen-binding fragment thereof comprises a heavy chain region comprising or consisting of SEQ ID No: 31 and a light chain region comprising or consisting of SEQ ID No: 35.

As demonstrated in FIG. 5, the antibodies of 51B12 and 63A11 share a high degree of sequence identity, which has enabled the inventors to identify the amino acid residues that are believed to be important for retaining anti-ZIP12 activity and have enabled them to generate a consensus sequence.

Accordingly, preferably the antibody or antigen-binding fragment thereof may comprise a CDR-H1 domain of SEQ ID No: 36, which is provided herein, as follows:
[SEQ ID No: 36]

Thus, preferably the antibody or antigen-binding fragment thereof comprises a CDR-H1 domain comprising or consisting of a sequence as substantially set out in SEQ ID No: 36, or a variant or fragment thereof, in which X can be any amino acid. Preferably, X is H or Y.

The antibody or antigen-binding fragment thereof may comprise a CDR-H2 domain of SEQ ID No: 37, which is provided herein, as follows:

[SEQ ID No: 37]
YISSGX¹X²X³IX⁴YADTVKG

Thus, preferably the antibody or antigen-binding fragment thereof comprises a CDR-H2 domain comprising or consisting of a sequence as substantially set out in SEQ ID No: 37, or a variant or fragment thereof, in which X¹, X², X³ and X⁴ may be any amino acid. Preferably, X¹ may be S or G. Preferably, X² may be S or T. Preferably, X³ may be A or T, and X⁴ may be F or Y.

The antibody or antigen-binding fragment thereof may comprise a CDR-H3 domain of SEQ ID No: 5, which is provided herein, as follows:

[SEQ ID No: 5]
WTNLYAMDY

Thus, preferably the antibody or antigen-binding fragment thereof comprises a CDR-H3 domain comprising or consisting of a sequence as substantially set out in SEQ ID No: 5, or a variant or fragment thereof.

The antibody or antigen-binding fragment thereof may comprise a variable heavy chain domain of SEQ ID No: 38, which is provided herein as follows:

[SEQ ID No: 38]
EVQLVESGGGLVKPGGSRKLSCAASGFTFSDX¹GMHWVQAPEKGLEWVA
YISSGX²X³X⁴IX⁵YADTVKGRFTX⁶SRDNAKNTLFLQMTSLRSEDTAMY
X⁷CARWTNLYAMDYWGQGTSVTVSS

Preferably, therefore, the antibody or antigen-binding fragment, or a fragment or variant thereof, comprises or consists of SEQ ID No: 38 in which X¹ to X⁷ can be any amino acid. Preferably, X¹ may be Y or H. Preferably, X² may be G or S. Preferably, X³ may be T or S, and X⁴ may be T or A, X⁵ may be Y or F. Preferably, X⁶ may be I or M. Preferably, X⁷ may be Y or F.

The antibody or antigen-binding fragment thereof may comprise a light chain CDR-L1 domain of SEQ ID No: 7, which is provided herein, as follows:

[SEQ ID No: 7]
RASKSVSTSGYSYMH

Thus, preferably the antibody or antigen-binding fragment thereof comprises a CDR-L1 domain comprising or consisting of a sequence as substantially set out in SEQ ID No: 7, or a variant or fragment thereof.

The antibody or antigen-binding fragment thereof may comprise a CDR-L2 domain of SEQ ID No: 8, which is provided herein, as follows:

[SEQ ID No: 8]
LASNLES

Thus, preferably the antibody or antigen-binding fragment thereof comprises a CDR-L2 domain comprising or consisting of a sequence as substantially set out in SEQ ID No: 8, or a variant or fragment thereof.

The antibody or antigen-binding fragment thereof may comprise a CDR-L3 domain of SEQ ID No: 39, which is provided herein, as follows:

[SEQ ID No: 39]
QHSREX¹PX²T

Thus, preferably the antibody or antigen-binding fragment thereof comprises a CDR-L3 domain comprising or consisting of a sequence as substantially set out in SEQ ID No: 39, or a variant or fragment thereof, in which X¹ and X² may be any amino acid. Preferably, X¹ may be L or V. Preferably, X² may be L or Y.

The antibody or antigen-binding fragment thereof may comprise a light chain variable sequence as set out in SEQ ID No: 40, which is provided herein, as follows:

[SEQ ID No: 40]
DIVLTQSPASLAVSLGQRATISCRASKSVSTSGYSYMHWYQQKPGQPPK
LLIYLASNLESGVPARFSGSGSGTDFTLNIHPVEEEDAATYYCQHSRE
X¹PX²TFGX³GTKLELK

Preferably, the antibody or antigen-binding fragment thereof comprises a light chain variable region comprising or consisting of a sequence as substantially set out in SEQ ID No: 40, or a variant or fragment thereof, in which X¹ and X² may be any amino acid. Preferably, X¹ may be L or V. Preferably, X² may be L or Y, X³ may be G or A.

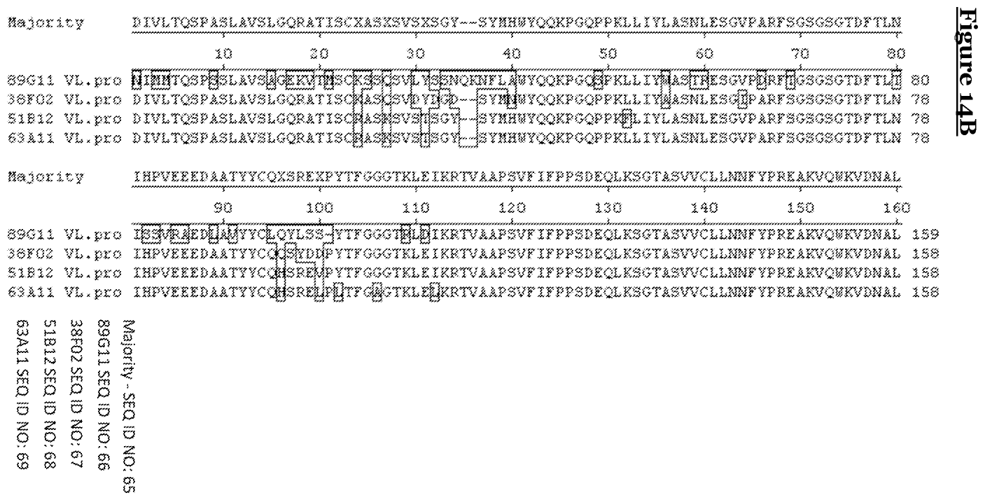

The antibody of the invention may comprise or consist of any sequence as defined in any one of FIGS. 14 to 19.

For example, the antibody or antigen-binding fragment thereof may comprise a heavy chain variable sequence comprising or consisting of a sequence as substantially set out in SEQ ID No: 69, 71 or 72, or a variant or fragment thereof. Thus, preferably, the antibody or antigen-binding fragment thereof may comprise a heavy chain variable sequence comprising or consisting of a sequence as substantially set out in SEQ ID No: 69, or a variant or fragment thereof. Preferably, the antibody or antigen-binding fragment thereof may comprise a heavy chain variable sequence comprising or consisting of a sequence as substantially set out in SEQ ID No: 71, or a variant or fragment thereof. Preferably, the antibody or antigen-binding fragment thereof may comprise a heavy chain variable sequence comprising or consisting of a sequence as substantially set out in SEQ ID No: 72, or a variant or fragment thereof.

For example, the antibody or antigen-binding fragment thereof may comprise a light chain variable sequence comprising or consisting of a sequence as substantially set out in SEQ ID No: 70, 73 or 74, or a variant or fragment thereof. Thus, preferably the antibody or antigen-binding fragment thereof may comprise a light chain variable sequence comprising or consisting of a sequence as substantially set out in SEQ ID No: 70, or a variant or fragment thereof. Preferably, the antibody or antigen-binding fragment thereof may comprise a light chain variable sequence comprising or consisting of a sequence as substantially set out in SEQ ID No: 73, or a variant or fragment thereof. Preferably, the antibody or antigen-binding fragment thereof may comprise a light chain variable sequence comprising or consisting of a sequence as substantially set out in SEQ ID No: 74, or a variant or fragment thereof.

In one embodiment, the antibody or antigen-binding fragment thereof may comprise a heavy chain variable sequence comprising or consisting of a sequence as substantially set out SEQ ID No: 69, 71 or 72 and a light chain variable sequence comprising or consisting of a sequence as substantially set out in SEQ ID No: 70, 73 or 74.

Thus, advantageously, the anti-ZIP12 activity of the antibody or antigen-binding fragment thereof according to the first aspect of the invention means that it has utility as a therapeutic agent in its own right, and may be used in the treatment, amelioration or prevention of a hypoxia-induced or hypoxia-associated condition, and in particular pulmonary hypertension.

Accordingly, in a second aspect of the invention, there is provided an antibody or an antigen-binding fragment thereof according to the first aspect, for use in therapy.

In a third aspect of the invention, there is provided an antibody or an antigen-binding fragment thereof according to the first aspect, for use in treating, preventing or ameliorating a hypoxia-related condition.

According to a fourth aspect of the invention, there is provided a method of treating, preventing or ameliorating a hypoxia-related condition in a subject, the method comprising administering, or having administered, to a patient in need of such treatment, a therapeutically effective amount of an antibody or antigen-binding fragment thereof according to the first aspect.

The hypoxia-related condition may be selected from the group consisting of: ischemic-reperfusion injury (IRI), cardiovascular disease, ischemic heart disease, ischemic brain condition, macular degeneration, ocular ischemic syndrome, ischemic optic neuropathy (ION), diabetic retinopathy, arthritis, inflammation, sepsis, sepsis-induced shock, renal disease, tissue fibrosis, gastrointestinal disease, neurodegenerative disease, respiratory distress syndrome, bronchopulmonary dysplasia, pulmonary hypertension, hypoxic pulmonary hypertension, severe pulmonary hypertension, COPD, idiopathic pulmonary fibrosis (IPF), diabetic retinopathy, diabetes, corneal neovascularization, pathogenic blood vessel growth, cancer and musculoskeletal disorder.

The inventors have demonstrated that the hypoxic core of tumours is a site of hypoxia-stimulated neoangiogenesis (Zhao et al 2015, The zinc transporter, ZIP12, regulates the pulmonary vascular response to chronic hypoxia, Nature, Vol:524, ISSN:0028-0836, Pages:356-360) and hypoxia is known as a universal hallmark of tumours and contributes towards resistance to radiation and chemotherapy. Thus, ZIP12, which the inventors have demonstrated to be upregulated in hypoxic conditions and is a key factor in disease progression, has been identified an as an ideal therapeutic target for cancer by the inventors. Inhibition of ZIP12 in the tumour microenvironment may restrict blood supply to the tumour and therefore restrict tumour growth and/or induce hypoxic tumour cell death.

Thus, in one embodiment, the hypoxia-related condition is cancer.

Preferably, the use or method in treating, preventing or ameliorating cancer comprises inhibiting angiogenesis. Preferably, the use or method comprises restricting blood supply to the tumour.

However, preferably, the hypoxia-related condition is pulmonary hypertension. Most preferably, the hypoxia-related condition is hypoxic pulmonary hypertension.

It will be appreciated that antibodies, fragments thereof according to the invention (collectively referred to herein as “agents”) may be used in a monotherapy (e.g. the use of an antibody or antigen binding fragment thereof alone), for treating, ameliorating or preventing hypoxia-related condition, and preferably pulmonary hypertension. Alternatively, agents according to the invention may be used as an adjunct to, or in combination with, known therapies for treating, ameliorating, or preventing hypoxia-related condition, preferably pulmonary hypertension, such as anticoagulants such as warfarin, diuretics, digoxin, endothelin receptor antagonists such as bosentan, ambrisentan and macitentan, phosphodiesterase 5 inhibitors such as sildenafil and tadalafil, prostaglandins such as epoprostenol, iloprost and treprostinil, soluble guanylate cyclase stimulators such as riociguat and calcium channel blockers nifedipine, diltiazem, nicardipine and amlodipine.

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin.

Agents and medicaments according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the antibodies and fragments (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the pulmonary hypertension. Additional factors depending on the particular subject being treated will result in a need to adjust dosages, including subject age, weight, gender, diet, and time of administration.

Generally, a daily dose of between 0.01 μg/kg of body weight and 100 mg/kg of body weight of agent according to the invention may be used for treating, ameliorating, or preventing coronavirus infection, depending upon which agent. More preferably, the daily dose of agent is between 1 μg/kg of body weight and 100 mg/kg of body weight, more preferably between 10 μg/kg and 10 mg/kg body weight, and most preferably between approximately 100 μg/kg and 10 mg/kg body weight.

The agent may be administered before, during or after onset of pulmonary hypertension. Daily doses may be given as a single administration (e.g. a single daily injection). Alternatively, the agent may require administration twice or more times during a day. As an example, agents may be administered as two (or more depending upon the severity of the pulmonary hypertension being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

In a fifth aspect of the invention, there is provided a pharmaceutical composition comprising an antibody or antigen-binding fragment thereof according to the first aspect, and optionally a pharmaceutically acceptable vehicle.

The pharmaceutical composition is preferably anti-hypertensive, i.e. a pharmaceutical formulation used in the therapeutic amelioration, prevention or treatment of pulmonary hypertension.

The invention also provides in a sixth aspect, a process for making the pharmaceutical composition according to the fifth aspect, the process comprising combining a therapeutically effective amount of an antibody or antigen-binding fragment thereof as defined in the first aspect, with a pharmaceutically acceptable vehicle.

The antibody or antigen-binding fragment thereof may be as defined with respect to the first aspect.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of the antibody or antigen-binding fragment thereof is any amount which, when administered to a subject, is the amount of agent that is needed to treat the pulmonary hypertension, or produce the desired effect.

For example, the therapeutically effective amount of antibody or fragment thereof used may be from about 0.1 ng/kg to about 100 mg/kg, and preferably from about 1 ng/kg to about 10 mg/kg. It is preferred that the amount of antibody or fragment is an amount from about 10 ng/kg to about 10 mg/kg, and most preferably from about 50 ng/kg to about 5 mg/kg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The invention also extends to methods for producing the antibody of the first aspect, and antibodies so produced.

In a seventh aspect, there is provided an antibody or antigen-binding fragment thereof obtained by a method comprising:-

(i) immunising a host organism with the ZIP12 extracellular domain, or a region of the ZIP12 protein, as defined in the first aspect; and

(ii) collecting an antibody or antigen-binding fragment thereof from the host.

The host may be a mammal, and may be a human, rabbit or mouse.

Preferably, the method comprises bleeding the host animal, and then preferably collecting the antibody or antigen-binding fragment thereof from the blood, most preferably blood serum. Preferably, the blood serum is passed through a gravity column with covalently bound peptide-support. Following washing, the antibody or antigen-binding fragment thereof is preferably eluted in buffer, which is preferably acidic buffer, and the solution may then be neutralized. The method may further comprise dialysis against a suitable buffer (e.g. PBS) and, optionally, lyophilisation.

In an eighth aspect of the invention, there is provided a polynucleotide sequence encoding the antibody or antigen binding fragment thereof as defined in the first aspect.

In a ninth aspect of the invention, there is provided an expression cassette comprising a polynucleotide sequence according to the eighth aspect.

The polynucleotide sequence encoding the antibody or antigen binding fragment thereof of the invention is preferably harboured in a recombinant vector, for example a recombinant vector for delivery into a host cell of interest to enable production of the antibody or antigen binding fragment thereof.

Accordingly, in a tenth aspect of the invention, there is provided a recombinant vector comprising the expression cassette according to the ninth aspect.

The vector encoding the antibody or antigen binding fragment thereof of the first aspect may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the antibody or antigen binding fragment thereof sequence forming the antibody or antigen binding fragment thereof of the first aspect.

Recombinant vectors encoding the antibody or antigen binding fragment thereof of the first aspect may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EFia, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene. The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion, calcium phosphate, cationic lipid-based lipofection, polymer or dendrimer-based methods or ballistic bombardment. Alternatively, vectors of the invention may be introduced directly into a host cell using a particle gun.

Alternatively, the delivery system may provide the polynucleotide to the host cell without it being incorporated in a vector. For instance, the nucleic acid molecule may be incorporated within a liposome or virus particle. Alternatively a “naked” polynucleotide may be inserted into a host cell by a suitable means e.g. direct endocytotic uptake.

In an eleventh aspect of the invention, there is provided a host cell comprising the polynucleotide sequence according to the eighth aspect, the expression cassette according to the ninth aspect, or the vector according to the tenth aspect.

The host cell may be a eukaryotic or prokaryotic host cell. Preferably, the host cell is a eukaryotic host cell. More preferably, the host cell is a mammalian host cell such as NSo murine myeloma cells, PER.C6® human cells, Human embryonic kidney 293 cells or Chinese hamster ovary (CHO) cells. Most preferably, the host cell is a CHO cell.

In a twelfth aspect, there is provided a method of preparing the antibody or antigen binding fragment according to the first aspect, the method comprising:

a) introducing, into a host cell, the vector of the tenth aspect; and

b) culturing the host cell under conditions to result in the production of the antibody or antigen binding fragment according to the first aspect.

The host cell of step a) may be a eukaryotic or prokaryotic host cell. Preferably, the host cell is a eukaryotic host cell. More preferably, the host cell is a mammalian host cell such as NSo murine myeloma cells, PER.C6® human cells, Human embryonic kidney 293 cells or Chinese hamster ovary (CHO) cells. Most preferably, the host cell is a CHO cell.

The method may further comprise (c) harvesting, centrifuging and/or filtering the cell culture media to obtain a cell culture supernatant comprising the antibody or antigen binding fragment thereof.

The method may further comprise (d) separating and purifying the antibody or antigen binding fragment thereof from the cell culture supernatant. Preferably, purification is performed by at least one chromatographic step.

Suitable chromatographic steps include affinity chromatography and/or ion exchange chromatography. Preferably, affinity chromatography is protein A chromatography. Ion exchange chromatography may be anionic exchange chromatography and/or cationic exchange chromatography.

Preferably, step (d) comprises separating and purifying the antibody or antigen binding fragment thereof from the cell culture supernatant by:
i) protein A chromatography;
ii) anionic exchange chromatography; and/or
iii) cationic exchange chromatography.

The method may further comprise (e) filtering the purified antibody or antigen binding fragment thereof resulting from step (d). Preferably, step (e) comprises virus filtration. Thus, preferably the purified antibody or antigen binding fragment thereof resulting from step (d) is filtered using a virus filtration membrane. Suitable membranes would be known to those skilled in the art.

As discussed herein, ZIP12 expression is increased in many cell types, including endothelial, smooth muscle and interstitial cells, in the remodelled pulmonary arterioles of rats, cows and humans susceptible to hypoxia-induced pulmonary hypertension. Thus, given that the antibodies of the invention are able to bind to the extracellular domain of ZIP12, the antibodies or antigen-binding fragments thereof may be used as a robust diagnostic tool by detecting the presence, and determining the concentration of, ZIP12.

Thus, in a thirteenth aspect, there is provided the antibody or antibody binding fragment of according to the first aspect, for use in diagnosis or prognosis.

According to a fourteenth aspect of the invention, there is provided the antibody or antibody binding fragment of according to the first aspect, for use in diagnosing or prognosing a hypoxia-related condition.

According to the fifteenth aspect, there is provided a method of diagnosing or prognosing a hypoxia-related condition in a subject, the method comprising detecting ZIP12 in a biological sample obtained from the subject with the antibody or antibody binding fragment of according to the first aspect.

Preferably, the hypoxia-related condition is selected from the group consisting of: ischemic-reperfusion injury (IRI), cardiovascular disease, ischemic heart disease, ischemic brain condition, macular degeneration, ocular ischemic syndrome, ischemic optic neuropathy (ION), diabetic retinopathy, arthritis, inflammation, sepsis, sepsis-induced shock, renal disease, tissue fibrosis, gastrointestinal disease, neurodegenerative disease, respiratory distress syndrome, bronchopulmonary displasia, pulmonary hypertension, hypoxic pulmonary hypertension, severe pulmonary hypertension, COPD, idiopathic pulmonary fibrosis (IPF), diabetic retinopathy, diabetes, corneal neovascularization, pathogenic blood vessel growth and musculoskeletal disorder.

Preferably, the hypoxia-related condition is cancer or pulmonary hypertension. Most preferably, hypoxia-related condition is pulmonary hypertension.

The method may be an in vitro or ex vivo method. Preferably, the method is an in vitro method.

The use or method may comprise determining the level of expression of ZIP12 in a subject, preferably wherein an increase in the concentration of ZIP12 in the biological sample when compared to a reference concentration from a healthy control population is indicative of pulmonary hypertension or a poor prognosis.

In one embodiment, the presence of ZIP12 in the lung may be considered indicative of pulmonary hypertension or a poor prognosis. The presence of ZIP12 may be detected by immunocytochemistry.

In one embodiment, a 1 fold increase of ZIP12 when compared to the reference from a healthy control population is indicative of pulmonary hypertension or a poor prognosis. In one embodiment, a 2 fold, 3 fold, 4 fold or 5 fold increase of ZIP12 when compared to the reference from a healthy control population is indicative of pulmonary hypertension or a poor prognosis. In one embodiment, a 10 fold, 50 fold or 100 fold increase of ZIP12 when compared to the reference from a healthy control population is indicative of pulmonary hypertension or a poor prognosis.

According to the sixteenth aspect of the invention, there is provided a kit for diagnosing a subject suffering from a hypoxia-related condition, or for providing a prognosis of the subject's condition, the kit comprising an antibody or antigen-binding fragment thereof according to the first aspect for detecting ZIP12 in a sample from a test subject.

The kit may further comprise instructions for use and/or a receptacle for obtaining a biological sample from a subject.

Preferably, the hypoxia-related condition is as defined in the fourteenth aspect, and is preferably pulmonary hypertension.

Prognosis may relate to determining the therapeutic outcome in a subject that has been diagnosed with pulmonary hypertension. Prognosis may relate to predicting the rate of progression or improvement and/or the duration of pulmonary hypertension in a subject, the probability of survival, and/or the efficacy of various treatment regimes. Thus, a poor prognosis may be indicative of pulmonary hypertension progression, low probability of survival and reduced efficacy of a treatment regime. A favourable prognosis may be indicative of pulmonary hypertension resolution, high probability of survival and increased efficacy of a treatment regime.

Preferably, the sample comprises a biological sample. The sample may be any material that is obtainable from a subject from which protein is obtainable.

The biological sample may be tissue or a biological fluid. The biological sample may be any material that is obtainable from the subject from which endothelial, smooth muscle and/or interstitial cells are obtainable. Furthermore, the sample may be blood, plasma, serum, spinal fluid, urine, sweat, saliva, tears, breast aspirate, breast milk, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, lymph, granuloma, cancer biopsy or combinations thereof.

The sample may be a liquid aspirate. For example, the sample may be bronchial alveolar lavage (BAL), ascites, pleural lavage, or pericardial lavage.

The sample may comprise blood, urine, tissue etc. In one preferred embodiment, the biological sample comprises a blood sample. The blood may be venous or arterial blood. Blood samples may be assayed immediately. Alternatively, the blood sample may be stored at low temperatures, for example in a fridge or even frozen before the method is conducted. Alternatively, the blood sample may be stored at room temperature, for example between 18 to 22 degrees Celsius, before the method is conducted. The blood sample may comprise comprises blood serum. The blood sample may comprise blood plasma. Preferably, however the detection is carried out on whole blood and most preferably the blood sample is peripheral blood.

The blood may be further processed before the use of the first aspect is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting.

Preferably, the sample may comprise an endothelial, smooth muscle and/or interstitial cell, preferably a pulmonary endothelial, smooth muscle and/or interstitial cell.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-75 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:- (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:- Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 75 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

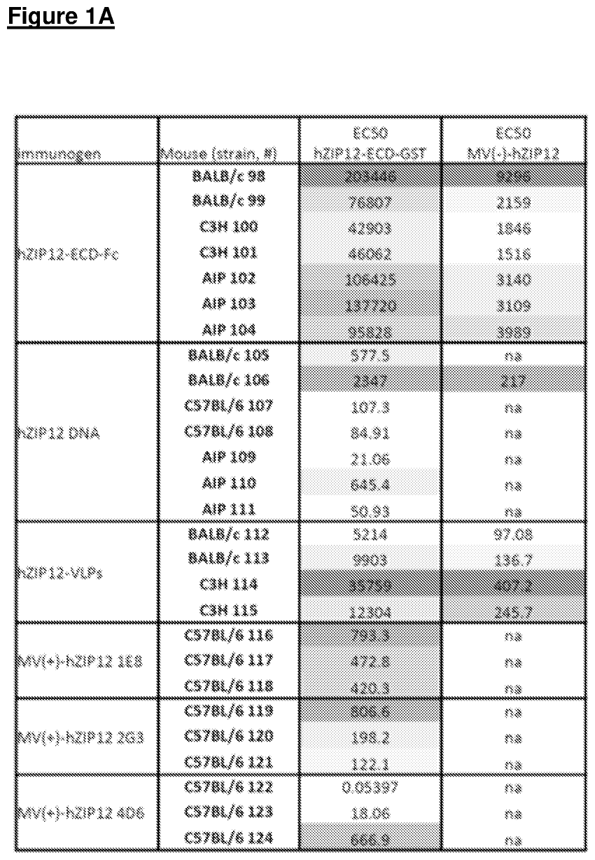

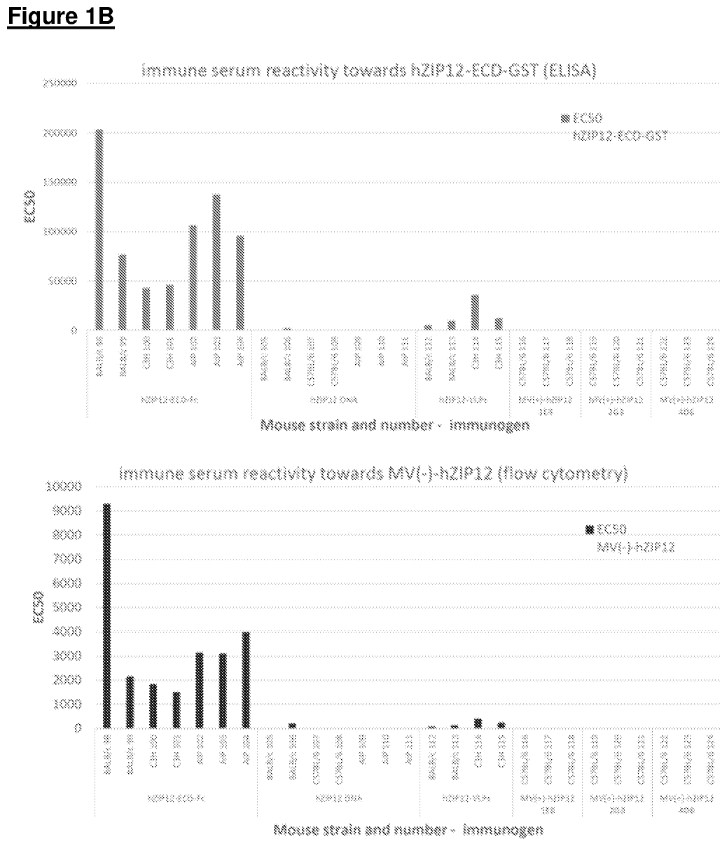

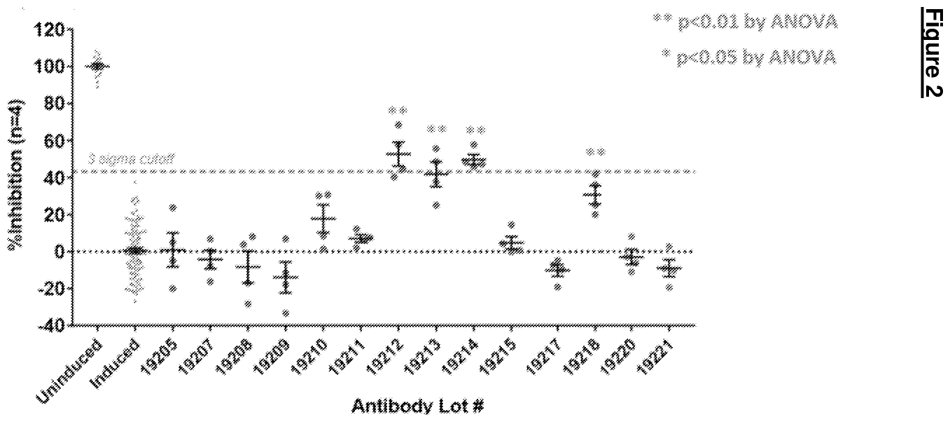

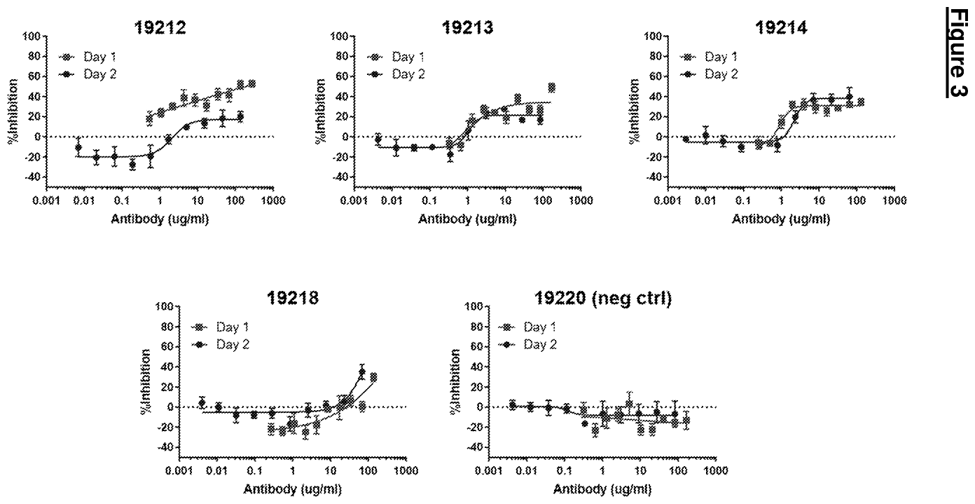

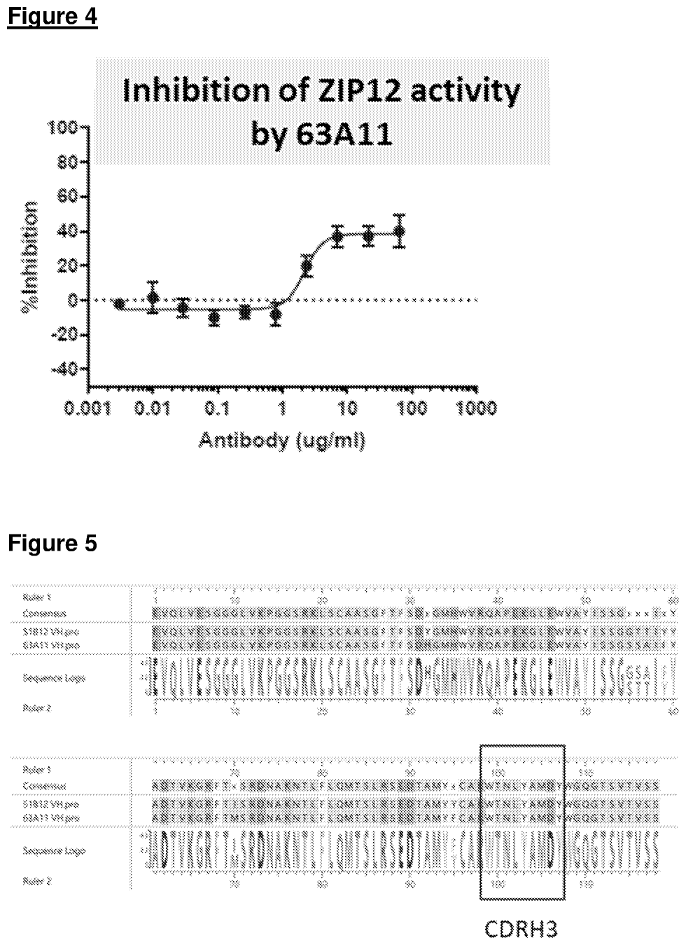

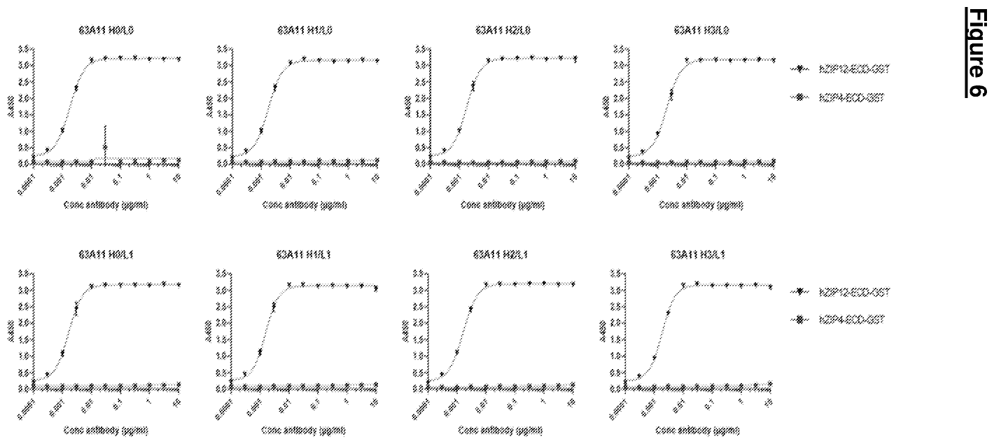

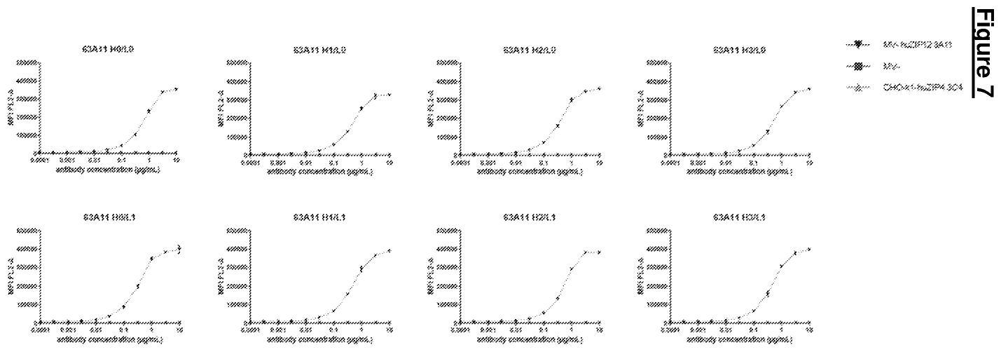

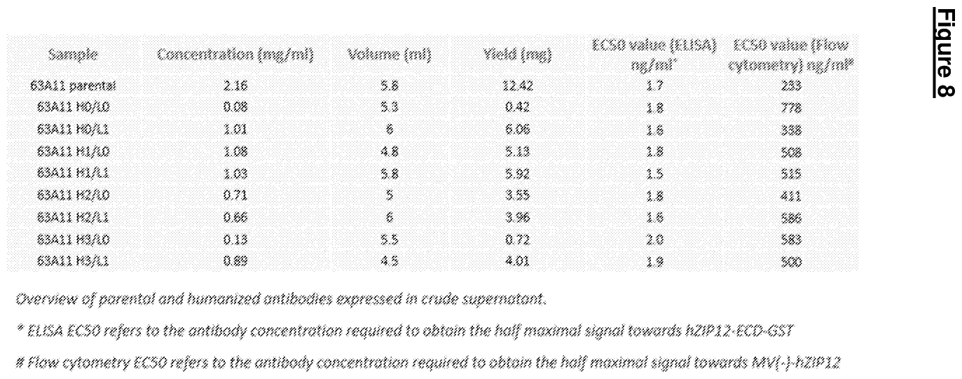

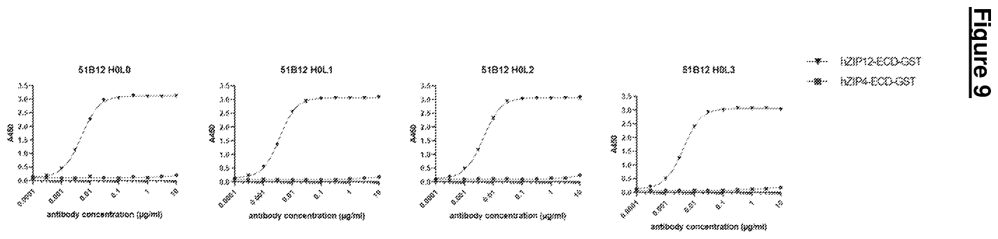

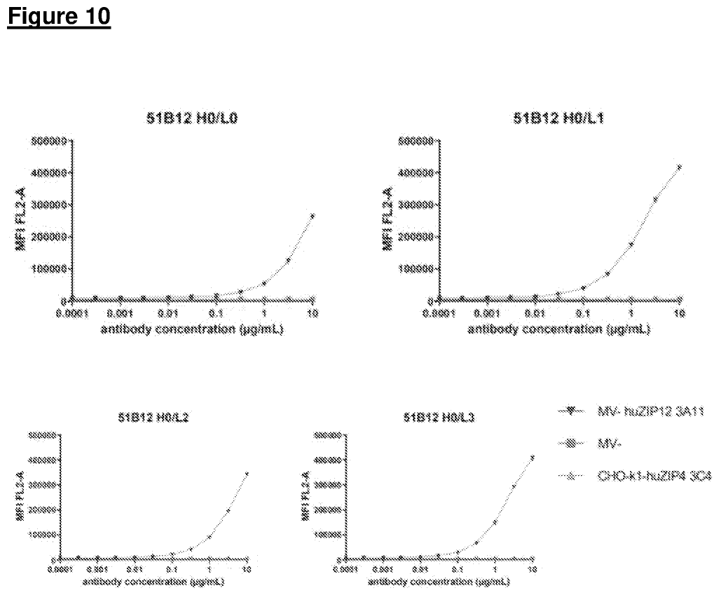

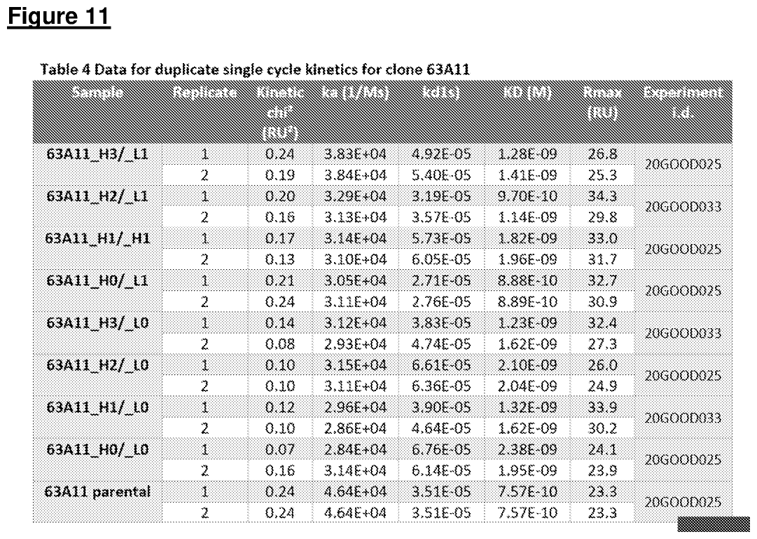

FIG. 1A provides a summary of the various ZIP12 immunogens used to stimulate immune responses in a number of mouse strains and the response of these mice to the immunogens, measured by ELISA and flow cytometry and shown graphically in FIG. 1B.

FIG. 2 shows an initial FLIPR screen of antibodies developed by the inventors demonstrating that a number of the antibodies demonstrate ZIP12 inhibitory activity. Data was calculated as maximum fluorescence signal—minimum fluorescence signal taken at 30 seconds prior to Zn addition (minimum signal) and 5 min after Zn+test agent addition (maximum signal).

FIG. 3 shows that the four most promising ZIP12 antibodies identified in the initial screen demonstrate dose-dependent inhibition of zinc uptake in a FLIPR assay. Antibody 19220 was used as a negative control. Antibody lots 19213 and 19214 correspond to the lead antibody hybridomas 63A11 (19214) and 51B12 (19213).

FIG. 4 shows the enlarged dose response graph of FIG. 3 for 63A11.

FIG. 5 shows an alignment between the 51B12 and 63A11 antibodies' VH sequences, demonstrating a high degree of sequence homology, including 100% sequence identity for the CDRH3 region. A consensus sequence produced from the alignment is also shown.

FIG. 6 shows 63A11 lineage: ELISA-based reactivity screening and shows that the parental antibody and also humanised heavy and light chains demonstrate anti-ZIP12 activity and are not reactive with ZIP4.

FIG. 7 shows 63A11 lineage flow cytometry-based reactivity screening and shows that the parental antibody and also humanised heavy and light chains demonstrate anti-ZIP12 activity and are not reactive with ZIP4.

FIG. 8 shows a summary of the properties of the 63A11 lineage antibodies.

FIG. 9 shows 51B12 lineage: ELISA-based reactivity screening and shows that the parental antibody and also humanised heavy and light chains demonstrate anti-ZIP12 activity and are not reactive with ZIP4.

FIG. 10 shows a summary of the properties of the 51B12 lineage antibodies.

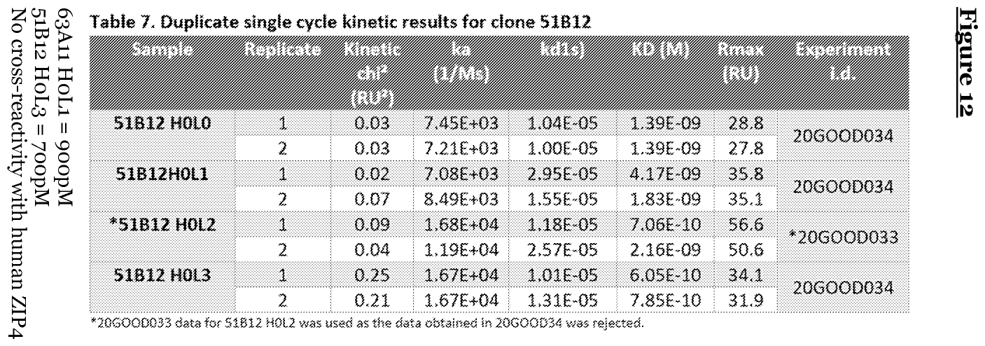

FIG. 11 shows biacore data for 63A11.

FIG. 12 shows biacore data for 51B12.

FIG. 13 is a table showing the sequences of four embodiments of the anti-ZIP12 antibody of the invention.

FIG. 14 shows an alignment and consensus sequence between the heavy chain sequences (a) and light chain sequences (b) of 89G11, 38F02, 51B12 and 63A11, all of which are embodiments of the invention. The amino acid sequence denoted as X in the majority sequence (SEQ ID No: 69) may comprise any amino acid sequence or one of those denoted in the same position of the sequences of 89G11, 38F02, 51B12 and 63A11.

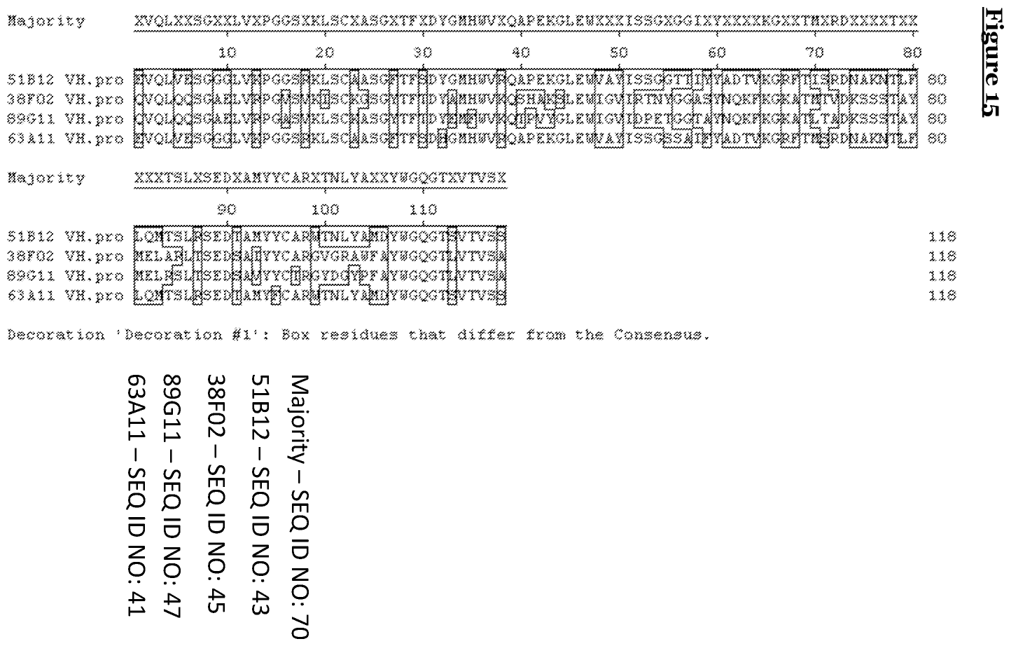

FIG. 15 shows an alignment and consensus sequence between the heavy chain variable sequences of 89G11, 38F02, 51B12 and 63A11, all of which form part of the invention. The amino acid sequence denoted as X in the majority sequence (SEQ ID No: 70) may comprise any amino acid sequence or the amino acids denoted in the same position of the sequences of 89G11, 38F02, 51B12 and 63A11.

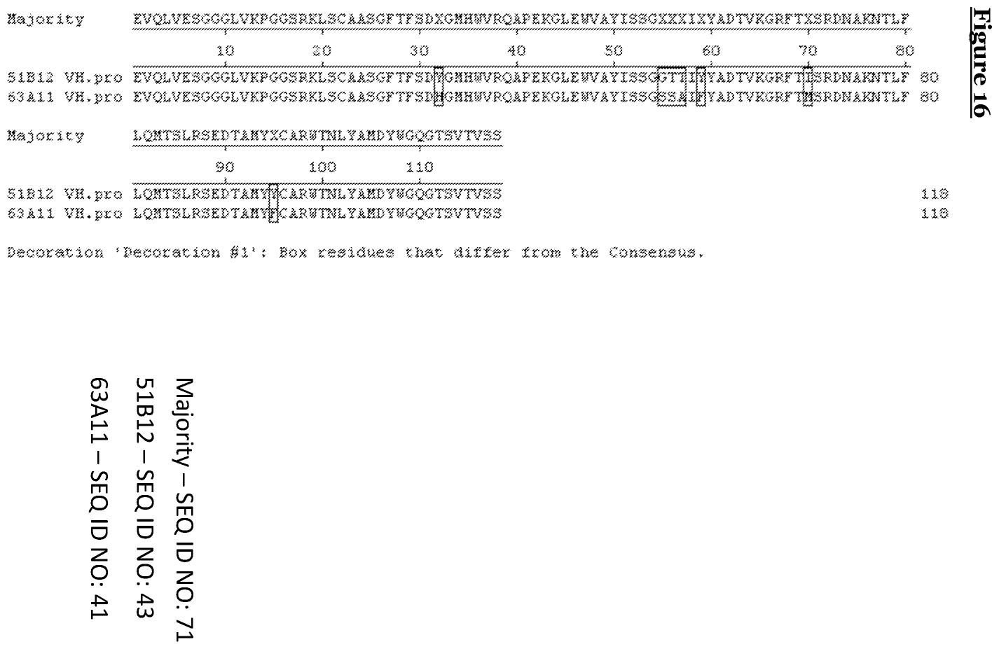

FIG. 16 shows an alignment and consensus sequence between the heavy chain variable sequences of 51B12 and 63A11, all of which form part of the invention. The amino acid sequence denoted as X in the majority sequence (SEQ ID No: 71) may comprise any amino acid sequence or the amino acids denoted in the same position of the sequences of 51B12 and 63A11.

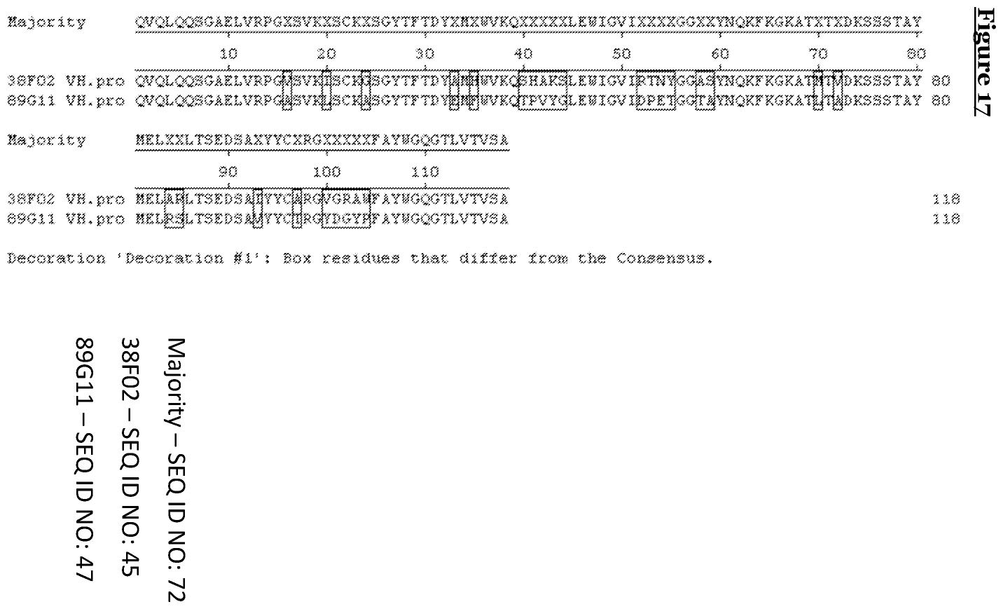

FIG. 17 shows an alignment and consensus sequence between the heavy chain variable sequences of 89G11 and 38F02, all of which form part of the invention. The amino acid sequence denoted as X in the majority sequence (SEQ ID No: 72) may comprise any amino acid sequence or the amino acids denoted in the same position of the sequences of 89G11 and 38F02.

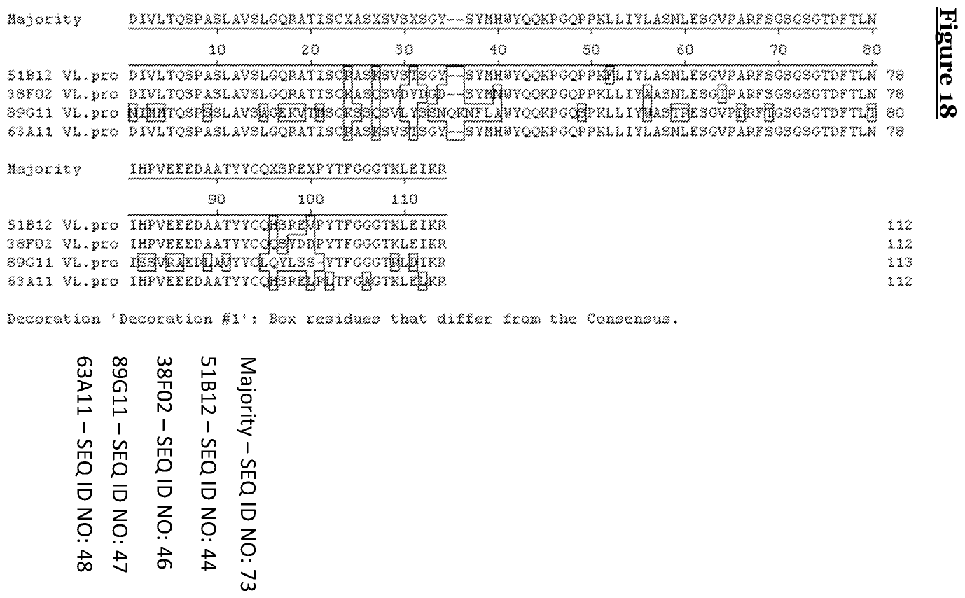

FIG. 18 shows an alignment and consensus sequence between the light chain variable sequences of 89G11, 38F02, 51B12 and 63A11, all of which form part of the invention. The amino acid sequence denoted as X in the majority sequence (SEQ ID No: 73) may comprise any amino acid sequence or the amino acids denoted in the same position of the sequences of 89G11, 38F02, 51B12 and 63A11.

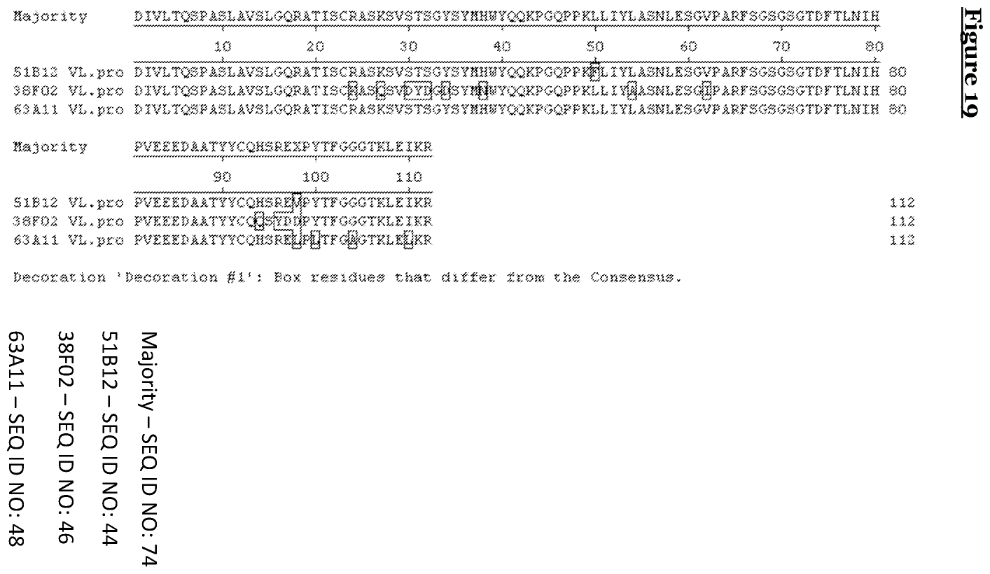

FIG. 19 shows an alignment and consensus sequence between the light chain variable sequences of 38F02, 51B12 and 63A11, all of which form part of the invention. The amino acid sequence denoted as X in the majority sequence (SEQ ID No: 74) may comprise any amino acid sequence or the amino acids denoted in the same position of the sequences of 38F02, 51B12 and 63A11.

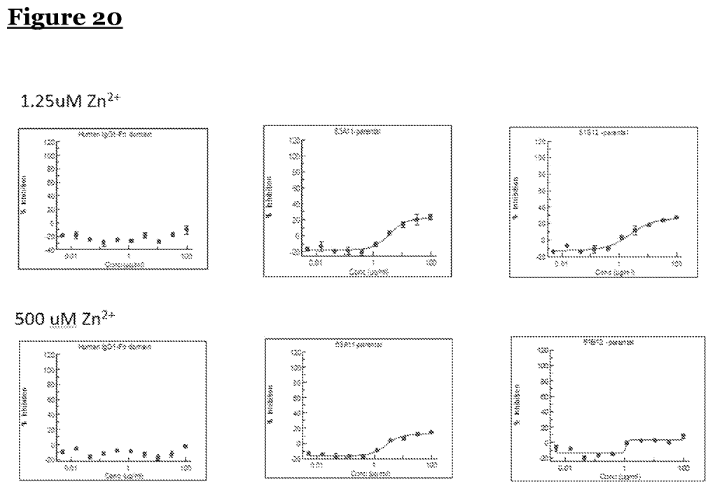

FIG. 20 shows that parent antibodies demonstrate dose-dependent inhibition of zinc uptake in a FLIPR assay at 1.25 uM and 500 uM zinc ions.

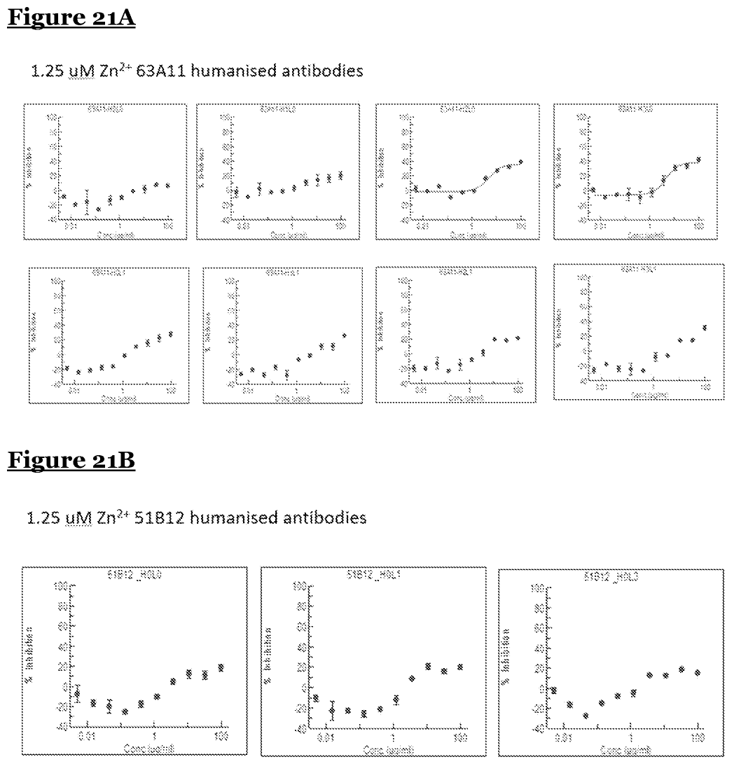

FIGS. 21A & B show that humanised antibodies (63A11 and 51B12) demonstrate dose-dependent inhibition of zinc uptake in a FLIPR assay.

### EXAMPLES

ZIP12 has been implicated in the pathogenesis of pulmonary hypertension, with increase in expression of ZIP12 associated with the condition. Therefore, the inventors set out to develop antibodies that are capable of specifically binding to and inhibiting the function of ZIP12, to provide improve the treatment and diagnosis of pulmonary hypertension.

### Example 1—Generation of ZIP12 Antibodies

Anti-human ZIP12 mAbs were generated from mice (BALB/c, C3H, AIC or C57BL/6) immunized with immunized with human ZIP12-ECD-Fc, human ZIP12-DNA, human ZIP12-VLPs or human ZIP12-ModiVacc (MV) stable cell lines. (FIG. 1A). Mice were immunised with antigen and boosted between one and three times at approximately one-month intervals.

ELISA-based serum reactivity screening towards hZIP12-ECD-Fc and hZIP12-ECD-GST (coated 5 μg/ml) and corresponding irrelevant control proteins human IgG and hZIP4-ECD-GST (coated at 1 and 5 μg/ml respectively). Sera from immune blood withdrawal at day 37 of VLP-immunized mice were also screened in an ELISA against hZIP12-VLPs and null-particles (coated 2 U/well). Serum samples were serially diluted (3-fold dilutions, 7 dilutions each starting from a 1:100 or 1:1,000 dilution) and detected with anti-mouse-IgG-HRP secondary antibody (1:5,000). Correct coating was confirmed using anti anti-ZIP12 (1:5,000), anti-ZIP4 (1:2,500) and detected with anti-rabbit IgG-HRP (1:5,000) or anti-human IgG-HRP (1:5,000). Flow cytometry-based serum reactivity screening towards MV(−)-hZIP12-3A11, MV(−) parental, MV(+) parental and CHO-k1-huZIP4-HA-3C4. Sera from mice immunized with MV(+)-hZIP12 stable cell lines were also screened in flow cytometry on MV(+)-hZIP12-1E8, MV(+)-hZIP12-2G3 and MV(+)-hZIP12-4D6 stable cell lines. Serum samples were serially diluted (3-fold dilutions, 7 dilutions each starting from a 1:100 dilution) and detected with anti-mouse-IgG-PE secondary antibody (1:300).

An overview of mouse strains and numbers, immunogens and serum titers, expressed as EC50 values (referring to the serum dilution yielding a half maximally signal) is presented in FIG. 1A. NA refers to not applicable; EC50 values could not be calculated due to too low response. 4 mice were selected for harvest and storage of splenocytes and bone marrow.

Hybridomas were either generated and cloned using the ClonaCell-HY hybridoma cloning kit (StemCell Technologies, Vancouver, BC) or using a conventional methods. In the conventional method, B cells from the spleens of the immunized animals were fused, by electrofusion using Modifuse technology, with Sp2/O myeloma cells in the presence of PEG (Sigma-Aldrich, St. Louis, MO). After overnight recovery, fused cells were plated at limiting dilution in 96-well plates and subjected to hypoxanthine-aminopterin thymidine selection. Hybridoma culture supernatants were examined for the presence of anti-ZIP12 antibodies by ELISA and flow cytometry (FIG. 1B). Immune serum activity was observed from hybridomas derived from BALB/c and C3H mice immunized with human ZIP12 virus-like particles and from BALB/c, AIP and C3H mice immunised with human ZIP12 extracellular domain fused to human Fc.

### Example 2—Hybridoma Variable Gene Sequencing

RNA was isolated from TRIzol samples. cDNA was generated and amplification of V-regions was completed according to standard procedures. PCR amplification of VH- and VL-gene fragments using MQR standard procedures or by using degenerate forward primers. Amplified V-genes were gel purified and cloned into human IgG1/IgK vectors using T4 ligase for DNA sequencing. Ligation mixes (˜25 ng vector) was transformed into chemocompetent E. coli XLi-Blue cells. Miniprep DNA was isolated from full-length insert containing clones. Isolated DNA (up to 10 VH and VL fragments per hybridoma) was sequenced and analysed using standard methods. Plasmids containing the correct genes were stored as glycerol stocks.

### Example 3—Generation of Chimeric Antibodies

The DNA expression constructs encoding the chimeric antibody were prepared using restriction sites for cloning into mammalian expression vectors as well as a human signal sequence. BsiWI and BsmI restriction sites were introduced to frame the variable domains containing the signal sequence for cloning into mammalian expression vectors containing the human γ 1 or human kappa constant regions.

The correct clones were confirmed using DNA sequencing. Plasmid DNA was transfected into HEK293 cells using FectoPro. Supernatants were harvested after ˜5 days. Antibody concentration was measured in culture supernatant the yield was calculated using Octet or ELISA. Antibodies were purified via protein A (Mab Select SuRe) resin and antibody concentrations were measured using Nanodrop. Antibody integrity and purity was confirmed using reducing SDS-PAGE. Antibody target reactivity was determined using ELISA and FACS.

### Example 4—Antibody Activity in a Zn2+FLIPR Assay

Generation of inducible ZIP12 cell lines: Inducible ZIP12 cell lines were generated by transfection of HEK293 or CHO cells with ZIP12 fused to a FLAG tag. Expression of ZIP12 was confirmed using PCR and Western blotting. In subsequent assays, cells were induced with tetracycline prior to screening.

Dye loading procedure: After cell induction and seeding, antibodies were prepared for pre-incubation. BSA was added to growth media at a final concentration of 0.1% and tetracycline at a concentration of 1 hg/ml. Antibodies were added at the desired concentration and incubated for either 4 or 24 hours at 37° C. Culture media was removed after ˜24 hours. EBSS, probenecid and Fluozin-3 were added to the reaction well and incubated in the dark for 1 hour. Dye solution was removed and Ca²⁺-free EBSS+2.5 mM probenecid was added and transferred to FLIPR.

FLIPR assay procedure: 10 μL, per well of assay buffer (Ca²⁺ free EBSS+2.5 mM probenecid) was added. 20 μL, per well of assay buffer was added with ZnSO₄ at 2× final desired concentration. Fluorescence was monitored for 10 minutes. % inhibition was calculated as maximum fluorescence signal—minimum fluorescence signal taken at 30 seconds prior to Zn addition (minimum signal) and 5 min after Zn+test agent addition (maximum signal).

### Example 5—Humanisation of ZIP12 Antibodies

(i) Cloning of the Humanised 51B12 and 63A11 Variants

The DNA expression constructs encoding the humanised antibody variants were prepared de novo by build-up of overlapping oligonucleotides including restriction sites for cloning into mammalian expression vectors as well as a human signal sequence. Hindlll and Spel restriction sites were introduced to frame the VH domain containing the signal sequence for cloning into mammalian expression vectors containing the human γ 1constant region. Hindlll and BsiWI restriction sites were introduced to frame the VL domain containing the signal sequence for cloning into mammalian expression vector containing the human kappa constant region.
(ii) Expression of the Recombinant 51B12 and 63A11 Antibodies (Including Antibody Quantification)

Expression plasmids encoding the heavy and light chains respectively were transiently co-transfected into HEK 293 6E cells and expressed at small scale to produce antibody. Antibodies were quantified by ELISA. ELISA plates were coated with anti human IgG (Sigma 13382) at img/ml and blocked with blocking solution (4% BSA in Tris buffered saline). Various dilutions of the tissue culture supernatants were added and the plate was incubated for ihour at room temperature. Dilutions of a known standard antibody were also added to the plate. The plate was washed in TBST and binding was detected by the addition of a peroxidise labelled anti human kappa light chain antibody (Sigma A71 64) at a dilution of 1/1000 in blocking solution. The plate was incubated for 1 hour at room temp before washing in TBST. The plate was developed by addition of OPD substrate (Sigma P9187) and colour development stopped by addition of 2M H2SO4. Absorbance was measured at 490 nm and a standard curve plotted using data for the known standard dilutions. The standard curve was used to estimate the concentration of antibody in the tissue culture supernatants. Larger scale antibody preparations were purified using protein A and concentrations were measured using a Nanodrop (Thermo Scientific).

### Example 6—ELISA Experiment Showing Binding of Humanised 63A11 to Recombinant Human ZIP12 Protein

Humanised 63A11 antibodies were tested for binding to human ZIP12 extracellular domain (expressed as a GST fusion protein). Human ZIP12 extracellular domain was coated to ELISA plates and the plates were blocked using BSA to reduce non-specific binding. Humanised antibodies were added in a concentration range from 10 ug/ml to 0.1 ug/ml to the human ZIP12 coated ELISA plates. Any bound humanised antibody was detected using anti-human IgG HRP conjugated secondary antibody as appropriate. HRP substrate (TMB) was added to develop the ELISA. This showed that 63A11 humanised antibodies bind to recombinant human ZIP12 in an ELISA assay. Results are shown in FIG. 6 and FIG. 8.

### Example 7—Humanised Antibodies Bind to ZIP12 Transfected Cells

Binding of humanised 63A11 to ZIP12 transfected cells as determined by FACS Human ZIP12 expressing transfectant cells were stained with humanised variants of 63A11 designated H0L0, H0L1, H1L0, H1L1, H2L0, H2L1, H3L0 and H3L1 at varying concentrations for 20 minutes at RT. Cells were then washed with FACS buffer (PBS+0.5% BSA+0.1% sodium azide) to remove unbound antibody. Cells were incubated with a secondary PE labelled anti-human IgG antibody for 1 hour at 4° C. in the dark and then washed with FACS buffer to remove unbound antibody. Cells were analysed by FACS and mean fluorescence intensity (MFI) values measured to determine binding. Results showed that all antibodies tested bound to human ZIP12 expressing transfectant cells in a dose dependent manner. Results are shown in FIG. 7 and FIG. 8.

### Example 8—ELISA Experiment Showing Binding of Humanised 51B12 Antibodies to Recombinant Human ZIP12 Protein

Humanised 51B12 antibodies were tested for binding to human ZIP12 extracellular domain (expressed as a GST fusion). Human ZIP12 extracellular domain was coated to ELISA plates and the plates were blocked using BSA to reduce non-specific binding. Humanised antibodies were added in a concentration range from 10 ug/ml to 0.1 ug/ml to the human ZIP12 coated ELISA plates. Any bound humanised antibody was detected using anti-human IgG HRP conjugated secondary antibody as appropriate. HRP substrate (TMB) was added to develop the ELISA. This showed that 51B12 humanised antibodies bind to recombinant human ZIP12 in an ELISA assay. Results are shown in FIG. 9.

### Example 9—Binding of Humanised 51B12 to ZIP12 Transfected Cells as Determined by FACS

Human ZIP12 expressing transfectant cells were stained with humanised variants of 51B12 designated H0L0, H0L1, H0L2 and H0L3 at varying concentrations for 20 minutes at RT. Cells were then washed with FACS buffer (PBS+0.5% BSA+0.1% sodium azide) to remove unbound antibody. Cells were incubated with a secondary PE labelled anti-human IgG antibody for 15 minutes at RT and then washed with FACS buffer to remove unbound antibody. Cells were analysed by FACS and mean fluorescence intensity (MFI) values measured to determine binding. Results showed that all antibodies tested bound to human ZIP12 expressing transfectant cells in a dose dependent manner. Results are shown in FIG. 10.

### Example 10—BIAcore Analysis of Anti-ZIP12 63A11 Humanised Constructs

A single concentration of hZIP12-ECD-GST (100 nM) was tested against the 63A11 clones. This stage confirmed the validity of the capture level of the antibodies and that we could observe binding of the hZIP12-ECD-GST fusion protein before proceeding with the assay development. The results also confirmed that the regeneration conditions (i.e. complete removal of hZIP12-ECD-GST from the surface by injection of a solution of 10 mM Glycine pH1.5 and 10 mM NaOH) were suitable with a complete return to baseline.

Protein A was immobilised on a Series S protein A sensor chip (Cyvita) by primary amine coupling and this surface was then used to capture the antibody molecules. All measurements were carried out at 25° C. and compartment temperature was held at 10° C. to help maintain the stability of the reagents. Data was collected at 10 Hz. Single cycle experiments were performed in duplicate at 25° C. 2-fold dilution concentration series of huZIP12-ECD-GST and huZIP4-ECD-GST, ranging from 6.25 nM to 400 nM for 63A11 clones in running buffer (ixHBS-EP+pH7.4), were injected over the surface of the sensor surface at 30 μl/min with 120 sec contact time and 2100 sec dissociation time for the 63A11 clones. The sensor surface was regenerated between cycles with 10 mM Glycine pH 1.5 for 60 sec at 50 μl/min and 10 mM NaOH for 60 sec at 50 μl/min. The association rate constant (ka) and the dissociation rate constant (kd) were calculated from reference and blank subtracted sensorgrams by fitting a 1:1 binding model, using the Biacore Insight Evaluation software (version3.0.11.15423 Cytiva). Both runs identified 63A11 H0L1 as the best antibody in term of overall affinity to human ZIP12. Data generated from this experiment are presented in FIG. 11.

### Example 11—BIAcore Analysis of Anti-ZIP12 51B12 Humanised Constructs

Protein A was immobilised on a Series S protein A sensor chip (Cyvita) by primary amine coupling and this surface was then used to capture the antibody molecules. All measurements were carried out at 25° C. and compartment temperature was held at 10° C. to help maintain the stability of the reagents. Data was collected at 10 Hz. Single cycle experiments were performed in duplicate at 25° C. 2-fold dilution concentration series of huZIP12-ECD-GST and huZIP4-ECD-GST, ranging from 12.5 nM to 800 nM for 51B12 clones in running buffer (ixHBS-EP+pH7.4), were injected over the surface of the sensor surface at 30 μl/min with 120 sec contact time and 2520 sec dissociation time for 51B12 clones. The sensor surface was regenerated between cycles with iomM Glycine pH 1.5 for 60 sec at 50 μl/min and iomM NaOH for 60 sec at 50 μl/min. The association rate constant (ka) and the dissociation rate constant (kd) were calculated from reference and blank subtracted sensorgrams by fitting a 1:1 binding model, using the Biacore Insight Evaluation software (version3.0.11.15423 Cytiva). Both runs identified 51B12 H0L3 as the best antibody in term of overall affinity to human ZIP12. Data generated from this experiment are presented in FIG. 13.

### Discussion & Conclusions

The inventors have identified the extracellular region or domain of the ZIP12 protein as being key to its function and have therefore developed antibodies that are capable of binding to, and inhibiting, ZIP12 function. For example, as shown in FIGS. 2 and 3, the inventors have developed a large number of antibodies that they have demonstrated specially target the extracellular domain and inhibit ZIP12 function. Furthermore, as shown in FIGS. 6, 7 and 9 the inventors have demonstrated that not only do the antibodies inhibit ZIP12 function, they do not target the ZIP4 protein.

The inventors have previously demonstrated that ZIP12 is new therapeutic target for the treatment of the underlying disease mechanisms of hypoxia-related conditions, particularly pulmonary hypertension. Therefore, the antibodies developed by the invention have utility as therapeutic agents in their own right, and may be used in the treatment, amelioration or prevention of a hypoxia-induced or hypoxia-associated condition, and in particular pulmonary hypertension.

### Example 12—Antibody Activity in a Zn²⁺ FLIPR Assay (2)

Methods

Cells were seeded and ZIP12 expression was induced with 1 μg/ml of tetracycline. After 4-6 hours following cell induction and seeding the cells were prepared for pre-incubation with antibodies. Tetracycline was not added to induced controls. 25 μl/well of antibodies were added to the cell growth plates already containing 25 μl/well of media. The final BSA concentration was 0.1%, the final tetracycline concentration at 1 μg/ml, and the final antibody concentration is a 1:10 dilution of the stock. Serial dilutions of antibody were set up in media in a separate plate before adding to cell plate. Antibodies and cells were incubated together at 37° C. for between 1-72 hours before removal of cell culture medium after antibody pre-incubation. For 384-well plate, 10 μL/well EBSS+2.5 mM probenecid+5 μM FluoZin-3 was added as per manufactures instruction. After incubation at 1 hour in the dark, dye solution was removed and 10 μL/well Ca²⁺-free EBSS and 2.5 mM probenecid were added, and the plate was analysed.

At time 0, 10 μL, of assay buffer was added to each well. After 1 minute, 20 μL, of assay buffer with ZnSO₄ at twice the final desired concentration was added to each well. Fluorescence was monitored for 10 minutes. The data was calculated as maximum fluorescence signal—minimum fluorescence signal taken at 30 seconds prior to Zn addition (minimum signal) and 5 min after Zn+test agent addition (maximum signal). 15% Inhibition was calculated as =(1−(Test well−Plate low control)/(Plate high control−Plate low control))*100. Inhibition relative to maximal inhibition was also calculated.

Results

FIG. 20 shows that two promising ZIP12 antibodies identified in the initial screen demonstrate dose-dependent inhibition of zinc uptake in a FLIPR assay. An irrelevant human antibody with an identical Fc region was used as a negative control.

FIGS. 21A and 21B show humanised antibodies (i.e. 63A11 and 51B12) derived from the two promising ZIP12 antibodies identified in the initial screen. These demonstrate dose-dependent inhibition of zinc uptake in a FLIPR assay.

1. An antibody or antigen-binding fragment thereof that specifically binds to an extracellular region of ZIP12.

2. The antibody or antigen binding fragment thereof according to claim 1, wherein the antibody or antigen binding fragment thereof does not substantially bind to human ZIP4 and/or ZIP13.

3. The antibody or antigen binding fragment thereof according to either claim 1 or claim 2, wherein the antibody or antigen-binding fragment thereof binds to a region between amino acid positions 1 and 202 of ZIP12, as substantially set out in SEQ ID No: 1.

4. The antibody or antigen binding fragment thereof according to any preceding claim, wherein the antibody or antigen binding fragment binds to an epitope within a sequence comprising or consisting of a sequence as substantially set out in SEQ ID No: 2, or a variant or fragment thereof.

5. The antibody or antigen binding fragment thereof according to any preceding claim, wherein the antibody or antigen-binding fragment thereof binds to one or more amino acids in SEQ ID No: 2, or a fragment or variant thereof, preferably wherein the antibody or antigen-binding fragment thereof binds to one or more amino acid between amino acid positions 20 and 202 of SEQ ID No: 2, or either between (i) amino acid positions 20 and 104 of SEQ ID No: 2, or (ii) between amino acid positions 156 and 202 of SEQ ID No: 2, more preferably wherein the antibody or antigen-binding fragment thereof binds to any 5, to, 15, 20, 25, 30, 35, 40 or 45 amino acid sequence present in SEQ ID No: 2, or a variant or fragment thereof.

6. The antibody or antigen-binding fragment thereof according to any preceding claim, wherein antibody or antigen-binding fragment thereof is a monoclonal antibody or an antigen-binding fragment thereof.

7. The antibody or antigen-binding fragment thereof according to any preceding claim, wherein the antibody or antigen-binding fragment thereof comprises:
(i) a CDR-H1 domain comprising or consisting of a sequence as substantially set out in SEQ ID No: 36, or a variant or fragment thereof, in which X is any amino acid, optionally wherein X is H or Y;
(ii) a CDR-H2 domain comprising or consisting of a sequence as substantially set out in SEQ ID No: 37, or a variant or fragment thereof, in which X¹, X², X³ and X⁴ is any amino acid, optionally wherein X¹ is S or G; X² is S or T; X³ is A or T; and/or X⁴ is F or Y;
(iii) a CDR-H3 domain comprising or consisting of a sequence as substantially set out in SEQ ID No: 5, or a variant or fragment thereof;
(iv) a CDR-L1 domain comprising or consisting of a sequence as substantially set out in SEQ ID No: 7, or a variant or fragment thereof;
(v) a CDR-L2 domain comprising or consisting of a sequence as substantially set out in SEQ ID No: 8, or a variant or fragment thereof and/or
(v) a CDR-L3 domain comprising or consisting of a sequence as substantially set out in SEQ ID No: 39, or a variant or fragment thereof, in which X¹ and X² is any amino acid, optionally wherein X¹ is L or V; and/or X² is L or Y.

8. The antibody or antigen-binding fragment thereof according to any preceding claim, wherein the antibody or antigen-binding fragment thereof comprises a CDR-H1 domain comprising SEQ ID No: 3, a CDR-H2 domain comprising SEQ ID No: 4, a CDR-H3 domain comprising SEQ ID No: 5, a CDR-L1 domain comprising SEQ ID No: 7, a CDR-L2 domain comprising SEQ ID No: 8, and/or a CDR-L3 domain comprising SEQ ID No: 9, optionally wherein the antibody or antigen-binding fragment thereof comprises at least one, at least two, at least three, at least four, at least five, or at least six of the CDRs.

9. The antibody or antigen-binding fragment thereof according to any preceding claim, wherein the antibody or antigen-binding fragment thereof comprises a heavy chain variable region comprising or consisting of SEQ ID No: 41 and a light chain variable region comprising or consisting of SEQ ID No: 42, optionally wherein the antibody or antigen-binding fragment thereof comprises a heavy chain region comprising or consisting of SEQ ID No: 6 and a light chain region comprising or consisting of SEQ ID No: 10.

10. The antibody or antigen-binding fragment thereof according to any preceding claim, wherein the antibody or antigen-binding fragment thereof comprises a heavy chain region comprising or consisting of a sequence selected from a group consisting of: SEQ ID Nos: 49, 50, 51, and 52 and a light chain region comprising or consisting of SEQ ID No: 53 or 54.

11. The antibody or antigen-binding fragment thereof according to any one of claims 1 to 7, wherein the antibody or antigen-binding fragment thereof comprises a CDR-H1 domain comprising SEQ ID No: 11, a CDR-H2 domain comprising SEQ ID No: 12, a CDR-H3 domain comprising SEQ ID No: 13, a CDR-L1 domain comprising SEQ ID No: 15, a CDR-L2 domain comprising SEQ ID No: 16, and/or a CDR-L3 domain comprising SEQ ID No: 17, optionally wherein the antibody or antigen-binding fragment thereof comprises at least one, at least two, at least three, at least four, at least five, or at least six of the CDRs.

12. The antibody or antigen-binding fragment thereof according to any one of claims 1 to 7, wherein the antibody or antigen-binding fragment thereof comprises a heavy chain variable region comprising or consisting of SEQ ID No: 43 and a light chain variable region comprising or consisting of SEQ ID No: 44, optionally wherein the antibody or antigen-binding fragment thereof comprises a heavy chain region comprising or consisting of SEQ ID No: 14 and a light chain region comprising or consisting of SEQ ID No: 18.

13. The antibody or antigen-binding fragment thereof according to any one of claims 1 to 7, wherein the antibody or antigen-binding fragment thereof comprises a heavy chain region comprising or consisting of SEQ ID No: 55 and a light chain region comprising or consisting of a sequence selected from a group consisting of: SEQ ID Nos: 56, 57, 58 and 59.

14. The antibody or antigen-binding fragment thereof according to any one of claims 1 to 7, wherein the antibody or antigen-binding fragment thereof comprises a CDR-H1 domain comprising SEQ ID No: 20, a CDR-H2 domain comprising SEQ ID No: 21, a CDR-H3 domain comprising SEQ ID No: 22, a CDR-L1 domain comprising SEQ ID No: 24, a CDR-L2 domain comprising SEQ ID No: 25, and/or a CDR-L3 domain comprising SEQ ID No: 26, optionally wherein the antibody or antigen-binding fragment thereof comprises at least one, at least two, at least three, at least four, at least five, or at least six of the CDRs.

15. The antibody or antigen-binding fragment thereof according to any one of claims 1 to 7, wherein the antibody or antigen-binding fragment thereof comprises a heavy chain variable region comprising or consisting of SEQ ID No: 45 and a light chain variable region comprising or consisting of SEQ ID No: 46, optionally wherein the antibody or antigen-binding fragment thereof comprises a heavy chain region comprising or consisting of SEQ ID No: 23 and a light chain region comprising or consisting of SEQ ID No: 27.

16. The antibody or antigen-binding fragment thereof according to any one of claims 1 to 7, wherein the antibody or antigen-binding fragment thereof comprises a CDR-H1 domain comprising SEQ ID No: 28, a CDR-H2 domain comprising SEQ ID No: 29, a CDR-H3 domain comprising SEQ ID No: 30, a CDR-L1 domain comprising SEQ ID No: 32, a CDR-L2 domain comprising SEQ ID No: 33, and/or a CDR-L3 domain comprising SEQ ID No: 34, optionally wherein the antibody or antigen-binding fragment thereof comprises at least one, at least two, at least three, at least four, at least five, or at least six of the CDRs.

17. The antibody or antigen-binding fragment thereof according to any one of claims 1 to 7, wherein the antibody or antigen-binding fragment thereof comprises a heavy chain variable region comprising or consisting of SEQ ID No: 47 and a light chain variable region comprising or consisting of SEQ ID No: 48, optionally wherein the antibody or antigen-binding fragment thereof comprises a heavy chain region comprising or consisting of SEQ ID No: 31 and a light chain region comprising or consisting of SEQ ID No: 35.

18. An antibody or an antigen-binding fragment thereof according to any preceding claim, for use in therapy.

19. An antibody or an antigen-binding fragment thereof according to any one of claims 1 to 17, for use in treating, preventing or ameliorating a hypoxia-related condition.

20. An antibody or an antigen-binding fragment thereof for use according to claim 19, wherein the hypoxia-related condition is pulmonary hypertension or cancer.

21. A pharmaceutical composition comprising an antibody or antigen-binding fragment thereof according to any one of claims 1 to 17, and optionally a pharmaceutically acceptable vehicle.

22. An antibody or antigen-binding fragment thereof obtained by a method comprising:-
(i) immunising a host organism with the ZIP12 extracellular domain, or a region of the ZIP12 protein, as defined in any one of claims 1 to 17; and
(ii) collecting an antibody or antigen-binding fragment thereof from the host.

23. A polynucleotide sequence encoding the antibody or antigen binding fragment thereof as defined in any one of claims 1 to 17.

24. An expression cassette comprising a polynucleotide sequence according to claim 23.

25. A recombinant vector comprising the expression cassette according to claim 24.

26. A host cell comprising the polynucleotide sequence according to claim 23, the expression cassette according to claim 24, or the vector according to claim 25.

27. A method of preparing the antibody or antigen binding fragment according to any one of claims 1 to 17, the method comprising:
a) introducing, into a host cell, the vector of claim 25; and
b) culturing the host cell under conditions to result in the production of the antibody or antigen binding fragment according to any one of claims 1 to 17.

28. The antibody or antibody binding fragment of according to any one of claims 1 to 17, for use in diagnosis or prognosis.

29. The antibody or antibody binding fragment of according to any one of claims 1 to 17, for use in diagnosing or prognosing a hypoxia-related condition.

30. A method of diagnosing or prognosing a hypoxia-related condition in a subject, the method comprising detecting ZIP12 in a biological sample obtained from the subject with the antibody or antibody binding fragment according any one of claims 1 to 17.

31. A kit for diagnosing a subject suffering from a hypoxia-related condition, or for providing a prognosis of the subject's condition, the kit comprising an antibody or antigen-binding fragment thereof according to any one of claims 1 to 17 for detecting ZIP12 in a sample from a test subject

32. The use according to claim 29, the method according to claim 30 or the kit according to claim 2931 wherein the hypoxia-related condition is pulmonary hypertension or cancer.


SEQUENCE LISTING (75 sequences)

SEQ ID NO: 1 - protein, 691 aa, Homo sapiens
MCFRTKLSVSWVPLFLLLSRVFSTETDKPSAQDSRSRGSSGQPADLLQVLSAGDHPPHNHSRSLIKTLLEKTGCPRRRNGMQGDCNLCFEPDALLLIAGGNFEDQLREEVVQRVSLLLLYYIIHQEEICSSKLNMSNKEYKFYLHSLLSLRQDEDSSFLSQNETEDILAFTRQYFDTSQSQCMETKTLQKKSGIVSSEGANESTLPQLAAMIITLSLQGVCLGQGNLPSPDYFTEYIFSSLNRTNTLRLSELDQLLNTLWTRSTCIKNEKIHQFQRKQNNIITHDQDYSNFSSSMEKESEDGPVSWDQTCFSARQLVEIFLQKGLSLISKEDFKQMSPGIIQQLLSCSCHLPKDQQAKLPPTTLEKYGYSTVAVTLLTLGSMLGTALVLFHSCEENYRLILQLFVGLAVGTLSGDALLHLIPQVLGLHKQEAPEFGHFHESKGHIWKLMGLIGGIHGFFLIEKCFILLVSPNDKQGLSLVNGHVGHSHHLALNSELSDQAGRGKSASTIQLKSPEDSQAAEMPIGSMTASNRKCKAISLLAIMILVGDSLHNFADGLAIGAAFSSSSESGVTTTIAILCHEIPHEMGDFAVLLSSGLSMKTAILMNFISSLTAFMGLYIGLSVSADPCVQDWIFTVTAGMFLYLSLVEMLPEMTHVQTQRPWMMFLLQNFGLILGWLSLLLLAIYEQNIKI

SEQ ID NO: 2 - protein, 202 aa, Homo sapiens
MCFRTKLSVSWVPLFLLLSRVFSTETDKPSAQDSRSRGSSGQPADLLQVLSAGDHPPHNHSRSLIKTLLEKTGCPRRRNGMQGDCNLCFEPDALLLIAGGNFEDQLREEVVQRVSLLLLYYIIHQEEICSSKLNMSNKEYKFYLHSLLSLRQDEDSSFLSQNETEDILAFTRQYFDTSQSQCMETKTLQKKSGIVSSEGANE

SEQ ID NO: 3 - protein, 5 aa, Artificial Sequence
  63A11 CDR-H1
DHGMH

SEQ ID NO: 4 - protein, 17 aa, Artificial Sequence
  63A11 CDR-H2
YISSGSSAIFYADTVKG

SEQ ID NO: 5 - protein, 9 aa, Artificial Sequence
  63A11 CDR-H3
WTNLYAMDY

SEQ ID NO: 6 - protein, 448 aa, Artificial Sequence
  63A11 HC
EVQLVESGGGLVKPGGSRKLSCAASGFTFSDHGMHWVRQAPEKGLEWVAYISSGSSAIFYADTVKGRFTMSRDNAKNTLFLQMTSLRSEDTAMYFCARWTNLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 15 aa, Artificial Sequence
  63A11 CDR-L1
RASKSVSTSGYSYMH

SEQ ID NO: 8 - protein, 7 aa, Artificial Sequence
  63A11 CDR-L2
LASNLES

SEQ ID NO: 9 - protein, 9 aa, Artificial Sequence
  63A11 CDR-L3
QHSRELPLT

SEQ ID NO: 10 - protein, 218 aa, Artificial Sequence
  63A11 LC
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, Artificial Sequence
  51B12 CDR-H1
DYGMH

SEQ ID NO: 12 - protein, 17 aa, Artificial Sequence
  51B12 CDR-H2
YISSGGTTIYYADTVKG

SEQ ID NO: 13 - protein, 9 aa, Artificial Sequence
  51B12 CDR-H3
WTNLYAMDY

SEQ ID NO: 14 - protein, 448 aa, Artificial Sequence
  51B12 HC
EVQLVESGGGLVKPGGSRKLSCAASGFTFSDYGMHWVRQAPEKGLEWVAYISSGGTTIYYADTVKGRFTISRDNAKNTLFLQMTSLRSEDTAMYYCARWTNLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 15 aa, Artificial Sequence
  51B12 CDR-L1
RASKSVSTSGYSYMH

SEQ ID NO: 16 - protein, 7 aa, Artificial Sequence
  51B12 CDR-L2
LASNLES

SEQ ID NO: 17 - protein, 9 aa, Artificial Sequence
  51B12 CDR-L3
QHSREVPYT

SEQ ID NO: 18 - protein, 218 aa, Artificial Sequence
  51B12 LC
DIVLTQSPASLAVSLGQRATISCRASKSVSTSGYSYMHWYQQKPGQPPKFLIYLASNLESGVPARFSGSGSGTDFTLNIHPVEEEDAATYYCQHSREVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 647 aa, Homo sapiens
MASLVSLELGLLLAVLVVTATASPPAGLLSLLTSGQGALDQEALGGLLNTLADRVHCANGPCGKCLSVEDALGLGEPEGSGLPPGPVLEARYVARLSAAAVLYLSNPEGTCEDARAGLWASHADHLLALLESPKALTPGLSWLLQRMQARAAGQTPKTACVDIPQLLEEAVGAGAPGSAGGVLAALLDHVRSGSCFHALPSPQYFVDFVFQQHSSEVPMTLAELSALMQRLGVGREAHSDHSHRHRGASSRDPVPLISSSNSSSVWDTVCLSARDVMAAYGLSEQAGVTPEAWAQLSPALLQQQLSGACTSQSRPPVQDQLSQSERYLYGSLATLLICLCAVFGLLLLTCTGCRGVTHYILQTFLSLAVGALTGDAVLHLTPKVLGLHTHSEEGLSPQPTWRLLAMLAGLYAFFLFENLFNLLLPRDPEDLEDGPCGHSSHSHGGHSHGVSLQLAPSELRQPKPPHEGSRADLVAEESPELLNPEPRRLSPELRLLPYMITLGDAVHNFADGLAVGAAFASSWKTGLATSLAVFCHELPHELGDFAALLHAGLSVRQALLLNLASALTAFAGLYVALAVGVSEESEAWILAVATGLFLYVALCDMLPAMLKVRDPRPWLLFLLHNVGLLGGWTVLLLLSLYEDDITF

SEQ ID NO: 20 - protein, 5 aa, Artificial Sequence
  38F02 CDR-H1
DYAMH

SEQ ID NO: 21 - protein, 17 aa, Artificial Sequence
  38F02 CDR-H2
VIRTNYGGASYNQKFKG

SEQ ID NO: 22 - protein, 9 aa, Artificial Sequence
  38F02 CDR-H3
GVGRAWFAY

SEQ ID NO: 23 - protein, 448 aa, Artificial Sequence
  38F02 HC
QVQLQQSGAELVRPGVSVKISCKGSGYTFTDYAMHWVKQSHAKSLEWIGVIRTNYGGASYNQKFKGKATMTVDKSSSTAYMELARLTSEDSAIYYCARGVGRAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 15 aa, Artificial Sequence
  38F02 CDR-L1
KASQSVDYDGDSYMN

SEQ ID NO: 25 - protein, 7 aa, Artificial Sequence
  38F02 CDR-L2
AASNLES

SEQ ID NO: 26 - protein, 9 aa, Artificial Sequence
  38F02 CDR-L3
QQSYDDPYT

SEQ ID NO: 27 - protein, 218 aa, Artificial Sequence
  38F02 LC
DIVLTQSPASLAVSLGQRATISCKASQSVDYDGDSYMNWYQQKPGQPPKLLIYAASNLESGIPARFSGSGSGTDFTLNIHPVEEEDAATYYCQQSYDDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 5 aa, Artificial Sequence
  89G11 CDR-H1
DYEMF

SEQ ID NO: 29 - protein, 17 aa, Artificial Sequence
  89G11 CDR-H2
VIDPETGGTAYNQKFKG

SEQ ID NO: 30 - protein, 9 aa, Artificial Sequence
  89G11 CDR-H3
GYGDYPFAY

SEQ ID NO: 31 - protein, 448 aa, Artificial Sequence
  89G11 HC
QVQLQQSGAELVRPGASVKLSCKASGYTFTDYEMFWVKQTPVYGLEWIGVIDPETGGTAYNQKFKGKATLTADKSSSTAYMELRSLTSEDSAVYYCTRGYDGYPFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 15 aa, Artificial Sequence
  89G11 CDR-L1
KSSQLYSSNQKNFLA

SEQ ID NO: 33 - protein, 7 aa, Artificial Sequence
  89G11 CDR-L2
WASTRES

SEQ ID NO: 34 - protein, 8 aa, Artificial Sequence
  89G11 CDR-L3
LQYLSSYT

SEQ ID NO: 35 - protein, 219 aa, Artificial Sequence
  89G11 LC
NIMMTQSPSSLAVSAGEKVTMSCKSSQSVLYSSNQKNFLAWYQQKPGQSPKLLIYWASTRESGVPDRFTGSGSGTDFTLTISSVRAEDLAVYYCLQYLSSYTFGGGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 5 aa, Artificial Sequence
  CDR-H1 consensus sequence. X can be any amino acid. Preferably, X is H or Y.
  misc_feature (2)..(2): Xaa can be any naturally occurring amino acid
DXGMH

SEQ ID NO: 37 - protein, 17 aa, Artificial Sequence
  CDR-H2 consensus sequence; X1, X2, X3 and X4 may be any amino acid. Preferably, X1 may be S or G. Preferably, X2 may be S or T. Preferably, X3 may be A or T, and X4 may be F or Y.
  misc_feature (6)..(8): Xaa can be any naturally occurring amino acid
  misc_feature (10)..(10): Xaa can be any naturally occurring amino acid
YISSGXXXIXYADTVKG

SEQ ID NO: 38 - protein, 118 aa, Artificial Sequence
  VH consensus sequence. X1 to X7 can be any amino acid. Preferably, X1 may be Y or H. Preferably, X2 may be G or S. Preferably, X3 may be T or S, and X4 may be T or A, X5 may be Y or F. Preferably, X6 may be I or M. Preferably, X7 may be Y or F.
  misc_feature (32)..(32): Xaa can be any naturally occurring amino acid
  misc_feature (55)..(57): Xaa can be any naturally occurring amino acid
  misc_feature (59)..(59): Xaa can be any naturally occurring amino acid
  misc_feature (70)..(70): Xaa can be any naturally occurring amino acid
  misc_feature (95)..(95): Xaa can be any naturally occurring amino acid
EVQLVESGGGLVKPGGSRKLSCAASGFTFSDXGMHWVRQAPEKGLEWVAYISSGXXXIXYADTVKGRFTXSRDNAKNTLFLQMTSLRSEDTAMYXCARWTNLYAMDYWGQGTSVTVSS

SEQ ID NO: 39 - protein, 9 aa, Artificial Sequence
  CDR-L3 consensus sequence. X1 and X2 may be any amino acid. Preferably, X1 may be L or V. Preferably, X2 may be L or Y.
  misc_feature (6)..(6): Xaa can be any naturally occurring amino acid
  misc_feature (8)..(8): Xaa can be any naturally occurring amino acid
QHSREXPXT

SEQ ID NO: 40 - protein, 111 aa, Artificial Sequence
  VL consensus sequence. X1 and X2 may be any amino acid. Preferably, X1 may be L or V. Preferably, X2 may be L or Y, X3 may be G or A.
  misc_feature (98)..(98): Xaa can be any naturally occurring amino acid
  misc_feature (100)..(100): Xaa can be any naturally occurring amino acid
  misc_feature (104)..(104): Xaa can be any naturally occurring amino acid
DIVLTQSPASLAVSLGQRATISCRASKSVSTSGYSYMHWYQQKPGQPPKLLIYLASNLESGVPARFSGSGSGTDFTLNIHPVEEEDAATYYCQHSREXPXTFGXGTKLELK

SEQ ID NO: 41 - protein, 118 aa, Artificial Sequence
  63A11 VH
EVQLVESGGGLVKPGGSRKLSCAASGFTFSDHGMHWVRQAPEKGLEWVAYISSGSSAIFYADTVKGRFTMSRDNAKNTLFLQMTSLRSEDTAMYFCARWTNLYAMDYWGQGTSVTVSS

SEQ ID NO: 42 - protein, 111 aa, Artificial Sequence
  63A11 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 43 - protein, 118 aa, Artificial Sequence
  51B12 VH
EVQLVESGGGLVKPGGSRKLSCAASGFTFSDYGMHWVRQAPEKGLEWVAYISSGGTTIYYADTVKGRFTISRDNAKNTLFLQMTSLRSEDTAMYYCARWTNLYAMDYWGQGTSVTVSS

SEQ ID NO: 44 - protein, 111 aa, Artificial Sequence
  51B12 VL
DIVLTQSPASLAVSLGQRATISCRASKSVSTSGYSYMHWYQQKPGQPPKFLIYLASNLESGVPARFSGSGSGTDFTLNIHPVEEEDAATYYCQHSREVPYTFGGGTKLEIK

SEQ ID NO: 45 - protein, 118 aa, Artificial Sequence
  38F02 VH
QVQLQQSGAELVRPGVSVKISCKGSGYTFTDYAMHWVKQSHAKSLEWIGVIRTNYGGASYNQKFKGKATMTVDKSSSTAYMELARLTSEDSAIYYCARGVGRAWFAYWGQGTLVTVSA

SEQ ID NO: 46 - protein, 111 aa, Artificial Sequence
  38F02 VL
DIVLTQSPASLAVSLGQRATISCKASQSVDYDGDSYMNWYQQKPGQPPKLLIYAASNLESGIPARFSGSGSGTDFTLNIHPVEEEDAATYYCQQSYDDPYTFGGGTKLEIK

SEQ ID NO: 47 - protein, 118 aa, Artificial Sequence
  89G11 VH
QVQLQQSGAELVRPGASVKLSCKASGYTFTDYEMFWVKQTPVYGLEWIGVIDPETGGTAYNQKFKGKATLTADKSSSTAYMELRSLTSEDSAVYYCTRGYDGYPFAYWGQGTLVTVSA

SEQ ID NO: 48 - protein, 112 aa, Artificial Sequence
  89G11 VL
NIMMTQSPSSLAVSAGEKVTMSCKSSQSVLYSSNQKNFLAWYQQKPGQSPKLLIYWASTRESGVPDRFTGSGSGTDFTLTISSVRAEDLAVYYCLQYLSSYTFGGGTRLDIK

SEQ ID NO: 49 - protein, 448 aa, Artificial Sequence
  63A11 HC
QVQLVESGGGVVQPGRSLRLSCAASGFTFSDHGMHWVRQAPGKGLEWVAYISSGSSAIFYADTVKGRFTISRDNSKNTLYLQMNSLRAEDTAVYYCARWTNLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 448 aa, Artificial Sequence
  63A11 HC
QVQLVESGGGVVQPGRSLRLSCAASGFTFSDHGMHWVRQAPGKGLEWVAYISSGSSAIFYADTVKGRFTISRDNSKNTLYLQMNSLRAEDTAVYFCARWTNLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 448 aa, Artificial Sequence
  63A11 HC
QVQLVESGGGVVQPGRSLRLSCAASGFTFSDHGMHWVRQAPGKGLEWVAYISSGSSAIFYADTVKGRFTMSRDNSKNTLYLQMNSLRAEDTAVYYCARWTNLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 448 aa, Artificial Sequence
  63A11 HC
QVQLVESGGGVVQPGRSLRLSCAASGFTFSDHGMHWVRQAPGKGLEWVAYISSGSSAIFYADTVKGRFTMSRDNSKNTLYLQMNSLRAEDTAVYFCARWTNLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 218 aa, Artificial Sequence
  63A11 LC
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 218 aa, Artificial Sequence
  63A11 LC
DIVLTQSPDSLAVSLGERATINCRASKSVSTSGYSYMHWYQQKPGQPPKLLIYLASNLESGVPDRFSGSGSGTDFTLTISSLQAEDVAVYYCQHSRELPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 448 aa, Artificial Sequence
  51B12 HC
QVQLVESGGGVVQPGRSLRLSCAASGFTFSDYGMHWVRQAPGKGLEWVAYISSGGTTIYYADTVKGRFTISRDNSKNTLYLQMNSLRAEDTAVYYCARWTNLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 218 aa, Artificial Sequence
  51B12 LC
DIVMTQSPDSLAVSLGERATINCRASKSVSTSGYSYMHWYQQKPGQPPKLLIYLASNLESGVPDRFSGSGSGTDFTLTISSLQAEDVAVYYCQHSREVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 218 aa, Artificial Sequence
  51B12 LC
DIVLTQSPDSLAVSLGERATINCRASKSVSTSGYSYMHWYQQKPGQPPKLLIYLASNLESGVPDRFSGSGSGTDFTLTISSLQAEDVAVYYCQHSREVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 218 aa, Artificial Sequence
  51B12 LC
DIVMTQSPDSLAVSLGERATINCRASKSVSTSGYSYMHWYQQKPGQPPKFLIYLASNLESGVPDRFSGSGSGTDFTLTISSLQAEDVAVYYCQHSREVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 218 aa, Artificial Sequence
  51B12 LC
DIVLTQSPDSLAVSLGERATINCRASKSVSTSGYSYMHWYQQKPGQPPKFLIYLASNLESGVPDRFSGSGSGTDFTLTISSLQAEDVAVYYCQHSREVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 160 aa, Artificial Sequence
  VH consensus sequence
  misc_feature (1)..(1): Xaa can be any naturally occurring amino acid
  misc_feature (5)..(6): Xaa can be any naturally occurring amino acid
  misc_feature (9)..(10): Xaa can be any naturally occurring amino acid
  misc_feature (13)..(13): Xaa can be any naturally occurring amino acid
  misc_feature (18)..(18): Xaa can be any naturally occurring amino acid
  misc_feature (23)..(23): Xaa can be any naturally occurring amino acid
  misc_feature (27)..(27): Xaa can be any naturally occurring amino acid
  misc_feature (30)..(30): Xaa can be any naturally occurring amino acid
  misc_feature (38)..(38): Xaa can be any naturally occurring amino acid
  misc_feature (48)..(50): Xaa can be any naturally occurring amino acid
  misc_feature (55)..(55): Xaa can be any naturally occurring amino acid
  misc_feature (59)..(59): Xaa can be any naturally occurring amino acid
  misc_feature (61)..(64): Xaa can be any naturally occurring amino acid
  misc_feature (67)..(68): Xaa can be any naturally occurring amino acid
  misc_feature (71)..(71): Xaa can be any naturally occurring amino acid
  misc_feature (74)..(77): Xaa can be any naturally occurring amino acid
  misc_feature (79)..(83): Xaa can be any naturally occurring amino acid
  misc_feature (87)..(87): Xaa can be any naturally occurring amino acid
  misc_feature (91)..(91): Xaa can be any naturally occurring amino acid
  misc_feature (99)..(99): Xaa can be any naturally occurring amino acid
  misc_feature (105)..(106): Xaa can be any naturally occurring amino acid
  misc_feature (113)..(113): Xaa can be any naturally occurring amino acid
  misc_feature (118)..(118): Xaa can be any naturally occurring amino acid
XVQLXXSGXXLVXPGGSXKLSCXASGXTFXDYGMHWVXQAPEKGLEWXXXISSGXGGIXYXXXXKGXXTMXRDXXXXTXXXXXTSLXSEDXAMYYCARXTNLYAXXYWGQGTXVTVSXASTKGPSVFPLAPSSKSTSGGTAALGCLVKDYFPEPVTVSWN

SEQ ID NO: 61 - protein, 160 aa, Artificial Sequence
  89G11 VH
QVQLQQSGAELVRPGASVKLSCKASGYTFTDYEMFWVKQTPVYGLEWIGVIDPETGGTAYNQKFKGKATLTADKSSSTAYMELRSLTSEDSAVYYCTRGYDGYPFAYWGQGTLVTVSAASTKGPSVFPLAPSSKSTSGGTAALGCLVKDYFPEPVTVSWN

SEQ ID NO: 62 - protein, 160 aa, Artificial Sequence
  38F02 VH
QVQLQQSGAELVRPGVSVKISCKGSGYTFTDYAMHWVKQSHAKSLEWIGVIRTNYGGASYNQKFKGKATMTVDKSSSTAYMELARLTSEDSAIYYCARGVGRAWFAYWGQGTLVTVSAASTKGPSVFPLAPSSKSTSGGTAALGCLVKDYFPEPVTVSWN

SEQ ID NO: 63 - protein, 160 aa, Artificial Sequence
  51B12 VH
EVQLVESGGGLVKPGGSRKLSCAASGFTFSDYGMHWVRQAPEKGLEWVAYISSGGTTIYYADTVKGRFTISRDNAKNTLFLQMTSLRSEDTAMYYCARWTNLYAMDYWGQGTSVTVSSASTKGPSVFPLAPSSKSTSGGTAALGCLVKDYFPEPVTVSWN

SEQ ID NO: 64 - protein, 160 aa, Artificial Sequence
  63A11 VH
EVQLVESGGGLVKPGGSRKLSCAASGFTFSDHGMHWVRQAPEKGLEWVAYISSGSSAIFYADTVKGRFTMSRDNAKNTLFLQMTSLRSEDTAMYFCARWTNLYAMDYWGQGTSVTVSSASTKGPSVFPLAPSSKSTSGGTAALGCLVKDYFPEPVTVSWN

SEQ ID NO: 65 - protein, 158 aa, Artificial Sequence
  VL consensus sequence
  misc_feature (24)..(24): Xaa can be any naturally occurring amino acid
  misc_feature (27)..(27): Xaa can be any naturally occurring amino acid
  misc_feature (31)..(31): Xaa can be any naturally occurring amino acid
  misc_feature (94)..(94): Xaa can be any naturally occurring amino acid
  misc_feature (98)..(98): Xaa can be any naturally occurring amino acid
DIVLTQSPASLAVSLGQRATISCXASXSVSXSGYSYMHWYQQKPGQPPKLLIYLASNLESGVPARFSGSGSGTDFTLNIHPVEEEDAATYYCQXSREXPYTFGGGTKLEIKRTVAAPSVFIFPPSDEQLKSGTASVVCLLNNFYPREAKVQWKVDNAL

SEQ ID NO: 66 - protein, 159 aa, Artificial Sequence
  89G11 VL
NIMMTQSPSSLAVSAGEKVTMSCKSSQSVLYSSNQKNFLAWYQQKPGQSPKLLIYWASTRESGVPDRFTGSGSGTDFTLTISSVRAEDLAVYYCLQYLSSYTFGGGTRLDIKRTVAAPSVFIFPPSDEQLKSGTASVVCLLNNFYPREAKVQWKVDNAL

SEQ ID NO: 67 - protein, 158 aa, Artificial Sequence
  38F02 VL
DIVLTQSPASLAVSLGQRATISCKASQSVDYDGDSYMNWYQQKPGQPPKLLIYAASNLESGIPARFSGSGSGTDFTLNIHPVEEEDAATYYCQQSYDDPYTFGGGTKLEIKRTVAAPSVFIFPPSDEQLKSGTASVVCLLNNFYPREAKVQWKVDNAL

SEQ ID NO: 68 - protein, 158 aa, Artificial Sequence
  51B12 VL
DIVLTQSPASLAVSLGQRATISCRASKSVSTSGYSYMHWYQQKPGQPPKFLIYLASNLESGVPARFSGSGSGTDFTLNIHPVEEEDAATYYCQHSREVPYTFGGGTKLEIKRTVAAPSVFIFPPSDEQLKSGTASVVCLLNNFYPREAKVQWKVDNAL

SEQ ID NO: 69 - protein, 158 aa, Artificial Sequence
  63A11 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELKRTVAAPSVFIFPPSDEQLKSGTASVVCLLNNFYPREAKVQWKVDNAL

SEQ ID NO: 70 - protein, 118 aa, Artificial Sequence
  VH consensus sequence
  misc_feature (1)..(1): Xaa can be any naturally occurring amino acid
  misc_feature (5)..(6): Xaa can be any naturally occurring amino acid
  misc_feature (9)..(10): Xaa can be any naturally occurring amino acid
  misc_feature (13)..(13): Xaa can be any naturally occurring amino acid
  misc_feature (18)..(18): Xaa can be any naturally occurring amino acid
  misc_feature (23)..(23): Xaa can be any naturally occurring amino acid
  misc_feature (27)..(27): Xaa can be any naturally occurring amino acid
  misc_feature (30)..(30): Xaa can be any naturally occurring amino acid
  misc_feature (38)..(38): Xaa can be any naturally occurring amino acid
  misc_feature (48)..(50): Xaa can be any naturally occurring amino acid
  misc_feature (55)..(55): Xaa can be any naturally occurring amino acid
  misc_feature (59)..(59): Xaa can be any naturally occurring amino acid
  misc_feature (61)..(64): Xaa can be any naturally occurring amino acid
  misc_feature (67)..(68): Xaa can be any naturally occurring amino acid
  misc_feature (71)..(71): Xaa can be any naturally occurring amino acid
  misc_feature (74)..(77): Xaa can be any naturally occurring amino acid
  misc_feature (79)..(83): Xaa can be any naturally occurring amino acid
  misc_feature (87)..(87): Xaa can be any naturally occurring amino acid
  misc_feature (91)..(91): Xaa can be any naturally occurring amino acid
  misc_feature (99)..(99): Xaa can be any naturally occurring amino acid
  misc_feature (105)..(106): Xaa can be any naturally occurring amino acid
  misc_feature (113)..(113): Xaa can be any naturally occurring amino acid
  misc_feature (118)..(118): Xaa can be any naturally occurring amino acid
XVQLXXSGXXLVXPGGSXKLSCXASGXTFXDYGMHWVXQAPEKGLEWXXXISSGXGGIXYXXXXKGXXTMXRDXXXXTXXXXXTSLXSEDXAMYYCARXTNLYAXXYWGQGTXVTVSX

SEQ ID NO: 71 - protein, 118 aa, Artificial Sequence; identical to SEQ ID NO: 38
  VH consensus sequence
  misc_feature (32)..(32): Xaa can be any naturally occurring amino acid
  misc_feature (55)..(57): Xaa can be any naturally occurring amino acid
  misc_feature (59)..(59): Xaa can be any naturally occurring amino acid
  misc_feature (70)..(70): Xaa can be any naturally occurring amino acid
  misc_feature (95)..(95): Xaa can be any naturally occurring amino acid

SEQ ID NO: 72 - protein, 118 aa, Artificial Sequence
  VH consensus sequence
  misc_feature (16)..(16): Xaa can be any naturally occurring amino acid
  misc_feature (20)..(20): Xaa can be any naturally occurring amino acid
  misc_feature (24)..(24): Xaa can be any naturally occurring amino acid
  misc_feature (33)..(33): Xaa can be any naturally occurring amino acid
  misc_feature (35)..(35): Xaa can be any naturally occurring amino acid
  misc_feature (40)..(44): Xaa can be any naturally occurring amino acid
  misc_feature (52)..(55): Xaa can be any naturally occurring amino acid
  misc_feature (58)..(59): Xaa can be any naturally occurring amino acid
  misc_feature (70)..(70): Xaa can be any naturally occurring amino acid
  misc_feature (72)..(72): Xaa can be any naturally occurring amino acid
  misc_feature (84)..(85): Xaa can be any naturally occurring amino acid
  misc_feature (93)..(93): Xaa can be any naturally occurring amino acid
  misc_feature (97)..(97): Xaa can be any naturally occurring amino acid
  misc_feature (100)..(104): Xaa can be any naturally occurring amino acid
QVQLQQSGAELVRPGXSVKXSCKXSGYTFTDYXMXWVKQXXXXXLEWIGVIXXXXGGXXYNQKFKGKATXTXDKSSSTAYMELXXLTSEDSAXYYCXRGXXXXXFAYWGQGTLVTVSA

SEQ ID NO: 73 - protein, 112 aa, Artificial Sequence
  VL consensus sequence
  misc_feature (24)..(24): Xaa can be any naturally occurring amino acid
  misc_feature (27)..(27): Xaa can be any naturally occurring amino acid
  misc_feature (31)..(31): Xaa can be any naturally occurring amino acid
  misc_feature (94)..(94): Xaa can be any naturally occurring amino acid
  misc_feature (98)..(98): Xaa can be any naturally occurring amino acid
DIVLTQSPASLAVSLGQRATISCXASXSVSXSGYSYMHWYQQKPGQPPKLLIYLASNLESGVPARFSGSGSGTDFTLNIHPVEEEDAATYYCQXSREXPYTFGGGTKLEIKR

SEQ ID NO: 74 - protein, 112 aa, Artificial Sequence
  VL consensus sequence
  misc_feature (98)..(98): Xaa can be any naturally occurring amino acid
DIVLTQSPASLAVSLGQRATISCRASKSVSTSGYSYMHWYQQKPGQPPKLLIYLASNLESGVPARFSGSGSGTDFTLNIHPVEEEDAATYYCQHSREXPYTFGGGTKLEIKR

SEQ ID NO: 75 - protein, 371 aa, Homo sapiens
MPGCPCPGCGMAGPRLLFLTALALELLERAGGSQPALRSRGTATACRLDNKESESWGALLSGERLDTWICSLLGSLMVGLSGVFPLLVIPLEMGTMLRSEAGAWRLKQLLSFALGGLLGNVFLHLLPEAWAYTCSASPGGEGQSLQQQQQLGLWVIAGILTFLALEKMFLDSKEEGTSQAPNKDPTAAAAALNGGHCLAQPAAEPGLGAVVRSIKVSGYLNLLANTIDNFTHGLAVAASFLVSKKIGLLTTMAILLHEIPHEVGDFAILLRAGFDRWSAAKLQLSTALGGLLGAGFAICTQSPKGVVGCSPAAEETAAWVLPFTSGGFLYIALVNVLPDLLEEEDPWRSLQQLLLLCAGIVVMVLFSLFVD